(19) United States
(12) Patent Application Publication
(10) Pub. No.: US 2024/0410883 A1
(43) Pub. Date: Dec. 12, 2024

(54) THERMALLY RESPONSIVE PARTITIONS FOR DEVICES AND SYSTEMS AND METHODS OF USING SAME

(71) Applicant: University of Maryland, College Park, College Park, MD (US)

(72) Inventors: Ian WHITE, Washington, DC (US); Micaela EVERITT, Washington, DC (US); David BOEGNER, White Hall, MD (US)

(21) Appl. No.: 18/699,251

(22) PCT Filed: Oct. 11, 2022
(86) PCT No.: PCT/US2022/077930

§ 371 Date: Apr. 5, 2024

Related U.S. Application Data
(60) Provisional application No. 63/262,319, filed on Oct. 8, 2021.

(51) Int. Cl. G01N 33/543; G01N 21/64; G01N 33/569; G01N 33/58

(52) U.S. Cl. CPC G01N 33/54333; G01N 21/6428; G01N 33/56983; G01N 33/582; G01N 2021/6439; G01N 2333/165; G01N 2333/908; G01N 2469/20

(57) ABSTRACT

A method and test system for detecting the presence of an analyte in a sample comprising a binding region and a detecting region, where the binding region contains a plurality of magnetic beads attached to a plurality of first capture molecules that bind to an analyte of interest in the sample, and a plurality of second capture molecules having a detectable label attached thereto, where the second capture molecules bind to the analyte of interest to form a complex, where the complexes are moved through at least one liquefied aliphatic partition via a magnetic field into the detecting region that having a detection composition allows for detection and, optionally, for signal quantification.

### CROSS REFERENCE TO RELATED APPLICATIONS

This application claims priority to U.S. Provisional Application No. 63/262,319, filed on Oct. 8, 2021, the disclosure of which is incorporated herein by reference.

### STATEMENT REGARDING FEDERALLY SPONSORED RESEARCH

This invention was made with government support under R01GM130923A awarded by the National Institutes of Health (NIH). The government has certain rights in the invention.

### BACKGROUND OF THE DISCLOSURE

Despite decades of research, the commercial impact of point-of-care (PoC) diagnostics has been minimal, and the need for global health diagnostics remains unaddressed. The lack of translation from research labs to the field may be due to incorrect assumptions and prioritizations about the requirements for use at the point of care. While efforts to shrink sensors or automate functions through microfluidics aim to satisfy some of the requirements outlined by the World Health Organization's “ASSURED” criteria, often the user-friendly (U in ASSURED) point is overlooked, despite its importance. In the United States, the Food and Drug Administration requires that diagnostic tests seeking approval for use outside of the central lab (i.e., to receive a Clinical Laboratory Improvement Amendment (CLIA) waiver) must be performed without the need for precise manual steps or interventions. This implies that diagnostic solutions should be sample-to-answer (S2A) to enable deployment beyond the central lab. To be a viable S2A diagnostic, a system must be able to process raw samples (including whole blood), enable precise reagent transfer and mixing, and perform rinses, all without manual interventions, bulky robotics, or complex valve controls.

Today, one common method for PoC protein biomarker detection is the lateral flow immunoassay (LFIA). All reagents are incorporated and stabilized, implying that precise reagent transfers are not necessary. Furthermore, the wicking of the membrane enables automated sample movement without the need for fluidics equipment. When the biomarker is present, a sandwich assay forms at a specific location on a paper strip, producing a visible line. As LFIAs require no wash steps, they are significantly faster and more hands-off than their gold standard counterparts, enzyme-linked immunosorbent assays (ELISAs). Saliva-based LFIA tests for HIV were an initial commercial success, and multiple nasal-swab-based COVID-19 tests quickly emerged in 2020, further proving the technology. This technology has been developed into diagnostic assays to rapidly diagnose viruses in serum or virus transport medium such as ebolavirus and influenza. While serum or saliva samples can be applied directly to the LFIA, complex raw samples, like whole blood, require preparation before the test can be performed. Although some progress has been made to separate plasma from whole blood by integrating a membrane-based separator into the LFIA, there are no commercially available LFIAs for protein detection in whole blood that are S2A when using complex raw samples.

The field of microfluidics has long pursued point-of-care diagnostics under the premise that precise reagent transfer steps can be automated by microfluidics, while biosensors can be integrated into the microsystems. Several reports demonstrate a reduction in the number of steps through non-chemical-based bacterial/viral lysis methods, and alternative DNA/RNA purification methods. While a reduction in the number of steps represents progress, these approaches still require one or more precise reagent transfers.

A few microfluidic methods have demonstrated S2A capabilities, but these methods have downfalls, including the need for cost-prohibitive chips or external pumps with manual tubing exchanges.

Magnetofluidics has recently emerged as a means to automate sample preparation, and thus to provide hands-off assay operation in PoC diagnostics. In initial reports, ferrofluids were manipulated with magnets underneath the device surface to transport droplets of reagents along the top of a surface, enabling automatic sample preparation for nucleic acid amplification tests. The surface of the nanoparticles serves to bind molecules within a droplet (e.g., DNA from cell lysate) and transport them to other droplets of reagents (e.g. rinsing and elution solutions). While these implementations pioneered a new hands-free method of sample manipulation, the reagents are typically not enclosed and are thus susceptible to contamination, evaporation, and mechanical disturbance.

Therefore, new methods and devices for point of care diagnostics are needed. These new methods and devices can allow for the detection of an analyte within a raw sample through a sample to answer immunoassay that is low cost and does not require bulky equipment.

### SUMMARY OF THE DISCLOSURE

The present disclosure provides a method and device utilizing thermally responsive aliphatic partitions (TRAPS) that initially separate reagent mixtures and continue to separate reagent mixtures, when liquefied, to allow magnetic beads to be pulled through the liquefied partitions in a magnetofluidics assay.

Various embodiments of the present disclosure provide a method for detecting the presence of an analyte. The method may comprise contacting a sample suspected of containing the analyte together or separately with (1) a plurality of magnetic beads that have a plurality of first capture molecules having specific affinity for the analyte attached thereto, and (2) a plurality of second capture molecules having a detectable label attached thereto and the second capture molecule has a specific affinity for the analyte, in order to form complexes comprising the analyte bound to the first capture molecule attached to the magnetic bead and the second capture molecule bound to the analyte. This method further comprises separating the complexes from unbound second capture molecules by selectively moving the complexes through one or more aliphatic partitions via application of a magnetic field, wherein the aliphatic partition has a melting point from 40° C. to 65° C. This method may further comprise detecting and optionally quantifying the signal generated from the detectable labels of the second capture molecules of the separated complexes. The presence of the detectable signal may be indicative of the presence of the analyte and the magnitude of the signal may be indicative of the amount of the analyte in the sample.

According to various embodiments of the present disclosure, the sample may be a biological sample.

According to various embodiments of the present disclosure, the sample may be chosen from whole blood, blood fractions, plasma, serum, saliva, urine, stool, sweat, mucous, tears, breast milk, semen, tissue, placental tissue, conditioned medium, tissue culture medium, and bone marrow.

According to various embodiments of the present disclosure, the analyte may be an antibody or an antigen.

According to various embodiments of the present disclosure, the antibody may be directed to a pathogenic antigen (e.g., a microbial antigen, bacterial antigen, or viral antigen).

According to various embodiments of the present disclosure, a viral antigen may be associated with SARS-CoV-2, Hepatitis C, Epstein-Barr, Zika, Ebola, Herpes simplex, Norovirus, Influenza, or Chikungunya.

According to various embodiments of the present disclosure, the plurality of magnetic beads may have a longest linear dimension of 100 nm to 50,000 nm.

According to various embodiments of the present disclosure, the second capture molecule have horseradish peroxidase attached thereto.

According to various embodiments of the present disclosure, the detection composition may comprise a detection molecule that may be colorimetric, luminescent, or fluorescent.

According to various embodiments of the present disclosure, a colorimetric detection molecule may be 3,3′,5,5′-Tetramethylbenzidine (TMB).

According to various embodiments of the present disclosure, a luminescent detection molecule may be Luminol.

According to various embodiments of the present disclosure, a fluorescent detection molecule may be Amplex Red.

According to various embodiments of the present disclosure, the sample may be sequentially contacted with the plurality of magnetic beads and the plurality of second capture molecules.

According to various embodiments of the present disclosure, the sample may be rinsed after contacting the plurality of magnetic beads and prior to contacting the plurality of second capture molecules.

According to various embodiments of the present disclosure, the aliphatic partition may comprise one or more alkanes.

According to various embodiments of the present disclosure, one or more alkanes may be chosen from eicosane, docosane, hexacosane, heptacosane, nonococane, tetracosane, and octadecane.

According to various embodiments of the present disclosure, the magnetic field may be generated by a magnet.

According to various embodiments of the present disclosure, the magnet may be moving at a rate of 0.2 mm/s to 10 mm/s.

According to an embodiment of the present disclosure the detectable signal may be colorimetric or fluorescent.

An aspect of the present disclosure is a testing system. The testing system may comprise a test assembly. The test assembly may comprise an inlet configured to accept a sample, a plurality of regions, and at least one aliphatic partition disposed therein. Adjacent regions of the plurality of regions may be separated by at least one of the aliphatic partitions. The plurality of regions may include a binding region and a detecting region. The binding region may comprise a plurality of magnetic beads having a plurality of first capture molecules attached thereto, wherein the first capture molecules have a specific affinity for the analyte and a plurality of second capture molecules having a detectable label attached thereto, wherein the second capture molecule has a specific affinity for the analyte. The detecting region may comprise a detection composition that allows direct or indirect spectrophotometric measurement of the detectable label. The testing system may comprise a magnet configured to apply a magnetic field along a sequential path that may include the binding region and the detecting region. The plurality of magnetic beads may move sequentially along the sequential path upon application of a magnetic force from the magnet.

According to various embodiments of the present disclosure, the sample may be an antibody or an antigen.

According to various embodiments of the present disclosure, the antibody may be associated with SARS-CoV-2, Hepatitis C, Epstein-Barr, Zika, Ebola, Herpes simplex, Norovirus, Influenza, or Chikungunya.

According to various embodiments of the present disclosure, the aliphatic partition may comprise one or more alkanes.

According to various embodiments of the present disclosure, one or more alkanes may be chosen from eicosane, docosane, hexacosane, heptacosane, nonococane, tetracosane, and octadecane.

According to various embodiments of the present disclosure, at least one aliphatic partition, the binding region, and the detecting region may be arranged in a horizontal hydrophobic channel or a vertical hydrophobic channel.

According to various embodiments of the present disclosure, at least one aliphatic partition, the binding region, and the detecting region may be arranged in a horizontal hydrophilic channel or a vertical hydrophilic channel.

According to various embodiments of the present disclosure, at least one of the aliphatic partitions may be configured to separate adjacent regions/sub-regions (e.g., the binding region and the detecting region).

According to various embodiments of the present disclosure, the binding region may comprise a plurality of sub-regions, and each adjacent region/sub-region may be separated by at least one aliphatic partition.

According to various embodiments of the present disclosure, the plurality of sub-regions may comprise a first binding sub-region and a second binding sub-region.

According to various embodiments of the present disclosure, the first binding sub-region may comprise the plurality of magnetic beads attached to a plurality of first capture molecules having a specific affinity for the analyte.

According to various embodiments of the present disclosure, the second binding sub-region may comprise the plurality of second capture molecules attached to a detectable label where the second capture molecules have a specific affinity for the analyte.

According to various embodiments of the present disclosure, the test assembly may comprise a rinsing sub-region disposed between the aliphatic partition of the first binding sub-region and the aliphatic partition of the second binding sub-region.

According to various embodiments of the present disclosure, the magnet may be external to the test assembly.

According to various embodiments of the present disclosure, the magnet may be configured to move across the test assembly along the sequential path.

According to various embodiments of the present disclosure, the test assembly may move across a sequential path relative to a magnet.

According to various embodiments of the present disclosure, the magnet may be disposed on an end of the test assembly and the magnetic field may extend across the binding region and the detecting region sequentially.

### BRIEF DESCRIPTION OF THE FIGURES

For a fuller understanding of the nature and objects of the disclosure, reference should be made to the following detailed description taken in conjunction with the accompanying figures.

FIG. 1 shows a cartoon representation of a method embodiment for detecting the presence of an analyte, utilizing three distinct compartments and two aliphatic partitions.

FIG. 2 shows a diagram representation of a method for detecting the presence of an analyte.

FIG. 3 shows a schematic of sample-to-answer immunoassay from whole blood. The first zone houses magnetic beads functionalized with SARS-CoV-2 spike proteins that capture antibodies against the SARS-CoV-2 spike protein in the whole blood sample. As the cartridge is heated, the TRAPs liquefy, allowing an external magnet to pull the beads through the first TRAP to the second zone. HRP-conjugated secondary antibodies in this second zone label any antibodies captured by the bead. These complexes are pulled through rinses to shed any labels that are caught in the hydration layer of the beads before reaching the detection zone. The detection zone contains Amplex Red and hydrogen peroxide, which fluoresces in the presence of HRP.

FIG. 4 shows a top perspective view of a test assembly.

FIG. 5 shows photos of demonstration of capillary-based cap for automated blood collection. (A) First the capillary cap is placed on the cartridge, then (B) the capillary is placed in contact with the blood sample (e.g., droplet on a fingertip) and (C) blood is quickly (i.e. 60 seconds) wicked into the capillary until (D) a precise volume is reached. (E) The cartridge is tapped vertically to dispel blood into the cartridge and (F) the capillary top is replaced with the sealed top. A video of a whole blood sample being loaded into the cartridge can be viewed in the supplement.

FIG. 6 shows (A) 3D representation of a device with an alkane TRAP partitioning two liquids. (B) Hypothetical immunoassay using TRAPS. Alkane wax layers partition all reagents. When warmed, the partitions liquefy, enabling magnetic beads to be pulled from one reagent zone to the next, enabling sample-to-answer assays.

FIG. 7 shows a TRAP with yellow dyed water on top of eicosane on top of blue dyed water.

FIG. 8 shows a schematic illustration of a portable fluorescence reader.

FIG. 9 shows (A) a photograph of a melted TRAP in a vertical hydrophobic channel. (B) 2D schematic of a cross-section along the center of the channel parallel to the image plane in (A) before and after melting the TRAP. (C) Data showing TRAPs that remained intact (circle) and TRAPs that breached (triangles) as well as the threshold (dashed line) determined by a mathematical approximation.

FIG. 10 shows (A) a photograph of a melted TRAP in a horizontal hydrophobic channel. (B) 2D schematic of a cross-section along the center of the channel parallel to the image plane in (A) before and after melting the TRAP. (C) Data showing TRAPs that remained intact (circle) and TRAPs that breached (triangles) as well as the threshold (dashed line) determined by the mathematical approximation from the vertical hydrophobic case.

FIG. 11 shows (A) a photograph of a melted TRAP in a vertical hydrophilic channel. (B) 2D schematic of a cross-section along the center of the channel parallel to the image plane in (A) before and after melting the TRAP. (C) Data showing TRAPs that remained intact (circle) and TRAPs that breached (triangles) as well as the threshold (dashed line) determined by a mathematical approximation

FIG. 12 shows (A) a photograph of a melted TRAP in a horizontal hydrophilic channel. (B) 2D schematic of a cross-section along the center of the channel parallel to the image plane in (A) before and after melting the TRAP. (C) Data showing TRAPs that remained intact (green circle) and TRAPs that breached (red triangles) as well as the threshold (dashed line) determined by a mathematical approximation.

FIG. 13 shows (A) a schematic of the experiment in which a fluorescence measurement was taken before and after magnetic beads were transferred across a TRAP. (B) Data showing fluorescence values of the FAM side (green) and water side (blue) of the TRAP.

FIG. 14 shows (A) a schematic depicting that fewer magnetic beads and a thicker TRAP (top) will not bridge while more beads and a thinner TRAP (bottom) may bridge, causing the two liquids to mix. (B) Data showing which experimental combinations of plug length and bead mass caused TRAPs to remain intact (green circle) or bridge (red triangle). (C) Before (top) and after (bottom) photos of 64 μg of beads travelling through a 2 mm plug length with no TRAP bridging.

FIG. 15 shows fluorescence signals of antibody-bound beads that travelled through one, two, or three TRAPs as well as a no target control (NTC) which included beads with no antibodies that travelled through one TRAP. n=3.

FIG. 16 shows impact of wax and heat on antibody sandwiches. (N=3) (A) Fluorescence measured before and after pulling beads across liquefied alkane partition. (B) Antibody sandwiches were exposed to 60° C. to determine heat impact on stability.

FIG. 17 shows (A) removal of biomarker from blood. Spike protein antibody is captured on magnetic bead and pulled across liquefied alkane to produce fluorescence. (N=3). (B) detection of SARS-CoV-2 antibodies using the sample-to-answer platform. (N=3).

FIG. 18 shows a schematic of a portable fluorescence reader with built in heater. (A) The ArduCAM, LEDs, filter, and heater are housed in a 3D printed handheld device. (B) The device opens to separate the heater from the ArduCAM to insert an assay cartridge. (C) The ArduCAM is housed behind a longpass filter to detect fluorescence produced from a sample, which is excited by LEDs built into the device above the sample cartridge. The cartridge is placed on top of a polyimide heater to liquefy the TRAPs.

FIG. 19 shows photos of magnetic beads pulled through TRAPs in cartridge. The cartridge is heated to melt the TRAPs. Once liquefied, the magnetic beads are pulled through each of the permeable barriers. The cartridge is removed from heat and the TRAPs harden. A video of magnetic beads being pulled through TRAPs can be viewed in the supplement.

FIG. 20 shows fluorescence quantification of antibodies against SARS-CoV-2 spike protein in whole blood over 14 minutes. The heater is turned off at t=0 minutes, right after the magnetic beads reach the detection zone.

FIG. 21 shows (A) fluorescence quantification of antibodies against SARS-CoV-2 spike protein in whole blood using our sample-to-answer immunoassay and portable fluorescence reader (N=3). The detection limit is 84 μg/mL using the IUPAC definition. Error bars are +/−1 standard deviation. (B) Fluorescence quantification of antibodies against SARS-CoV-2 spike protein in whole blood using a bead-based immunoassay with manual rinse steps and benchtop plate reader (N=3). The detection limit is 68 μg/mL using the IUPAC definition. Error bars are +/−1 standard deviation.

FIG. 22 shows (A) application of the TRAP in which magnetic beads move biomarkers out of a blood layer through a melted (liquefied) alkane into a rinse zone. (B) Photo and schematic of a resin channel showing two dyed water layers separated by a TRAP.

FIG. 23 shows several combinations of plug length and channel width for vertical (left) and horizontal (right), hydrophobic (top), hydrophilic (bottom) channels. Circles=TRAP stayed, triangle=TRAP broke and dyes mixed.

FIG. 24 shows fluorescence measurements of the water layer (bottom marks on left plot) and fluorescence layer (top marks on left plot) along with their corresponding schematics of a channel before and after beads were pulled through a TRAP via a magnet.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Throughout this application, the use of the singular form encompasses the plural form and vice versa. For example, “a”, or “an” also includes a plurality of the referenced items, unless otherwise indicated.

As used herein, unless otherwise indicated, the term “aliphatic” refers to branched or unbranched hydrocarbon groups that, optionally, contain one or more degree(s) of unsaturation. Degrees of unsaturation can arise from, but are not limited to, cyclic aliphatic groups. For example, the aliphatic groups/moieties are a C₁₆ to C₄₀ aliphatic group, including all integer numbers of carbons and ranges of numbers of carbons therebetween (e.g., C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). Aliphatic groups include, but are not limited to, alkyl groups, alkene groups, and alkyne groups. The aliphatic group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, thioether groups, thioester groups, and the like, and combinations thereof.

As used herein, unless otherwise indicated, the term “alkyl group” refers to branched or unbranched saturated hydrocarbon groups. The alkyl group can be a C₁₆ to C₄₀ alkyl group, including all integer numbers of carbons and ranges of numbers of carbons there between (e.g., C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). The alkyl group can be unsubstituted or substituted with one or more substituents. Examples of substituents include, but are not limited to, various substituents such as, for example, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof.

This disclosure provides a method for detecting the presence of an analyte in a sample by the use of thermally responsive aliphatic partitions (TRAPs) and magnetic beads. The thermally responsive aliphatic partitions may operate in at least two distinct behavior modes: (1) as a removable partition for hands-free reagent mixing following melting, and (2) as a continual partition that separates assay regions while enabling magnetic beads to be pulled through following melting, to enable hands-free immune-magnetic assays. Also provided are devices that utilize a method of the present disclosure.

In an aspect, the present disclosure provides a method of detecting an analyte of a sample. In some embodiments, the analyte may be detected to determine the disease state of an individual (e.g., whether or not the individual has a viral infection or whether or not the individual possesses antibodies for a particular viral infection).

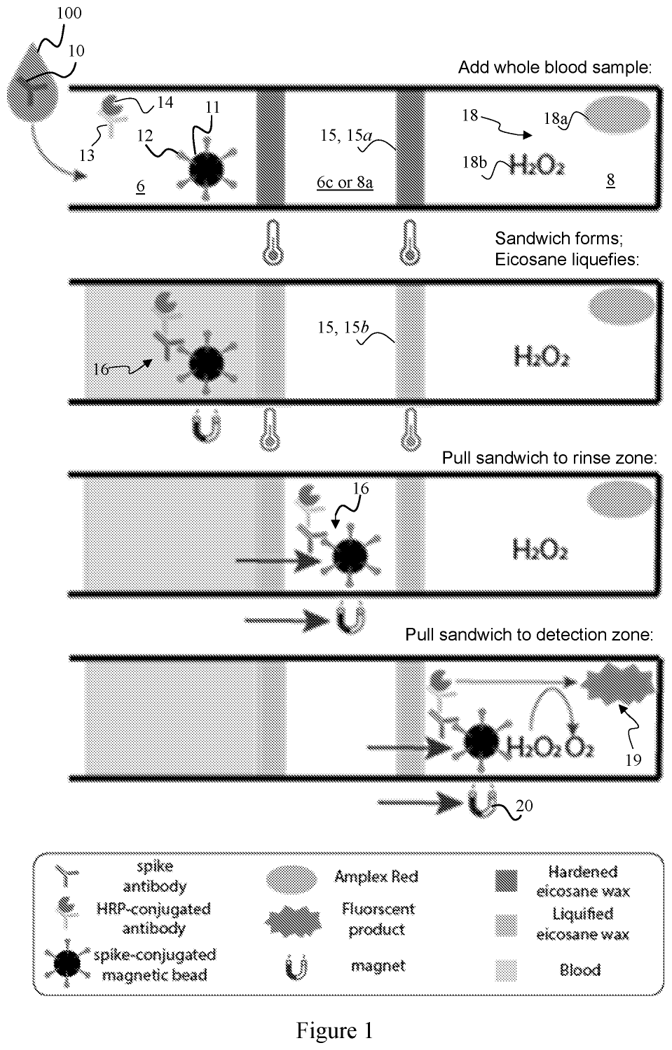

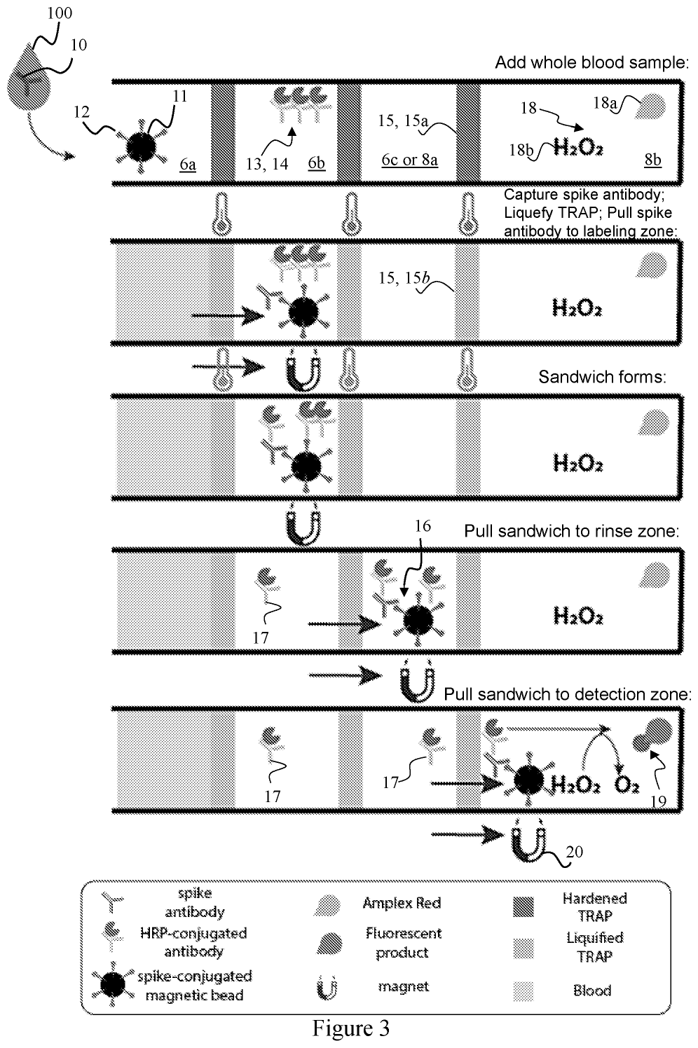

In various embodiments of a method for detecting the presence of an analyte in a raw sample may comprise using a thermally responsive aliphatic partition that separates adjacent assay regions while enabling magnetic beads to be moved into adjacent regions following melting of the aliphatic partition (FIG. 1). “Raw sample” may be used interchangeably with the term “sample.” “Melted partition” may be used interchangeably with the term “liquefied partition.” This embodiment may comprise the following steps:
contacting a raw sample, such as a blood sample 100, that comprises or is suspected of comprising the analyte of interest 10, with (1) a plurality of magnetic beads 11 that have a plurality of first capture molecules 12 attached thereto, where the first capture molecules 12 have a specific affinity for the analyte 10 within the sample (e.g., blood sample 100), and (2) a plurality of second capture molecules 13 having a specific affinity for the analyte 10 in order to form complexes 16. The second capture molecules have a detectable label 14 attached thereto. The complexes 16 comprise the analyte 10 bound to the first capture molecule 12 attached to the magnetic bead 11 and the second capture molecule 13 bound to the analyte 10;
heating the aliphatic partitions 15 to a temperature at or between 40° C. to 65° C. such that aliphatic partitions 15 liquefy. At temperatures below 40° C., the aliphatic partitions are in a solid state 15a and limit (e.g., restrict or prevent) the movement of the complexes 16, and at temperatures at or between 40° C. to 65° C., the aliphatic partitions are in a liquid state 15b;
separating the complexes 16 from unbound second capture molecules 17 (shown in FIG. 3) by selectively moving the complexes 16 through one or more liquefied aliphatic partitions 15b;
detecting and optionally quantifying the signal generated from the detectable labels 14 of the second capture molecules 13 of the separated complexes 16.

In various embodiments, the method comprises a heating step, wherein the one or more solid aliphatic partitions 15a are liquefied such that bound analyte may be moved through the liquefied aliphatic partitions 15b via application of a magnetic field.

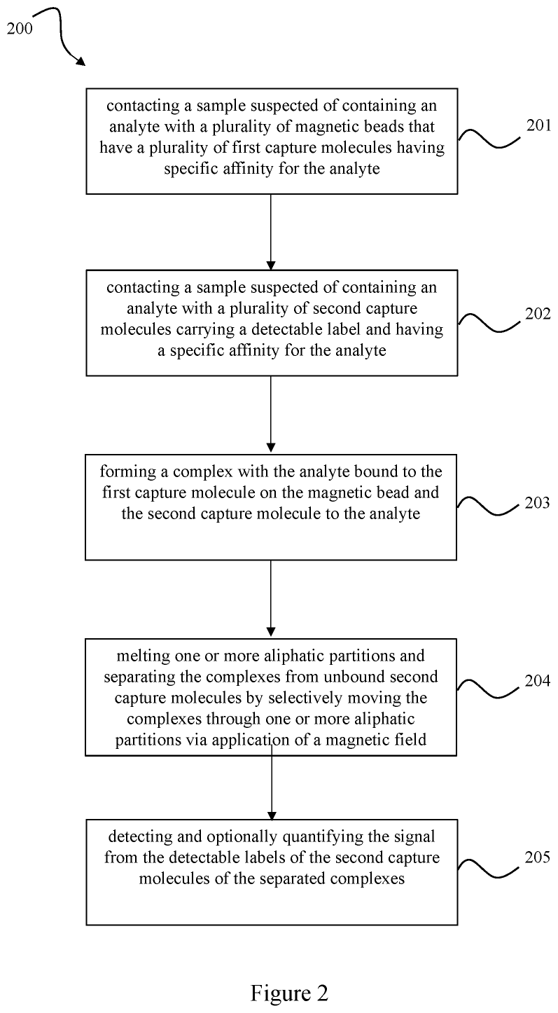

FIG. 2 displays a block diagram of the method disclosed herein. The method 200, may be broken into 5 distinct steps, 201, 202, 203, 204, and 205. Step 201 may comprise contacting a sample suspected of comprising an analyte with a plurality of magnetic beads that have a plurality of first capture molecules attached thereto, where the first capture molecules have a specific affinity for the analyte. Step 202 may comprise contacting a sample suspected of comprising an analyte with a plurality of second capture molecules having a detectable label attached thereto, where the second capture molecules have a specific affinity for the analyte. Step 203 may comprise forming a complex with the analyte bound to the first capture molecule and the second capture molecule to the analyte. Step 204 may comprise melting one or more aliphatic partitions separating adjacent regions and separating the complexes from unbound second capture molecules by selectively moving the complexes through one or more liquefied aliphatic partitions via application of a magnetic field. And step 205 may comprise detecting and optionally quantifying the signal from the detectable labels of the second capture molecules of the separated complexes.

The detectable label 14, on the second capture molecule 13, may be detected directly or indirectly. For example, as shown in FIGS. 1 and 3, the detectable label 14 may be indirectly detected via reaction with a detection molecule 18a or otherwise catalyze a reaction with a detection molecule 18a to generate a detectable signal. In other examples, the detectable label 14 is directly detectable via spectrophotometry. In various embodiments, the detectable label 14 reacts with a detection molecule 18a that may be colorimetric, luminescent, or fluorescent, to generate a signal 19 that is proportional (e.g., directly proportional) to the concentration of the analyte 10 within the sample 100. In various embodiments, the signal 19 may be compared to a control that does not comprise the detection molecule, detection label, or combination thereof.

As shown in FIGS. 1 and 3, various embodiments of the method comprise moving the sample through at least two regions, including a binding region 6 and a detecting region 8. The regions may further be divided into sub-regions. For example, the binding region 6 may be divided into one or more sub-regions. Examples of sub-regions include a first sub-region 6a comprising the first capture molecules 12 attached to magnetic beads 11, a second sub-region 6b comprising the second capture molecule attached to a detectable label, and one or more rinse regions 6c. For example, the binding region 6 may sequentially comprise the following sub-regions: a first binding sub-region 6a comprising the plurality of magnetic beads 11 and the first capture molecules 12, a rinse sub-region 6c, a second binding sub-region 6b comprising the second capture molecules 13 and the detectable label 14, where each sub-region is separated by an aliphatic partition 15. In various examples, the binding region 6 further comprises a second rinse sub-region 6c adjacent to the second-binding sub-region 6b and the sub-regions are separated by an aliphatic partition 15. In various embodiments, the binding region 6 comprises a binding sub-region comprising the plurality of magnetic beads 11, the first capture molecules 12, the second capture molecules 13, and the detectable label 14, and rinsing sub-region 6c, where the sub-regions are separated by an aliphatic partition 15. The detecting region 8 may optionally comprise one or more sub-regions. For example, the detecting region 8 may comprise a rinse sub-region 8a and a detecting sub-region 8b, where the detection sub-region 8b and rinse sub-region 8a are separated by an aliphatic partition 15. An aliphatic partition 15 would separate each region/sub-region.

The one or more rinse regions/sub-regions comprise an aqueous medium. In various other embodiments, the rinse region/sub-region may comprise water. In various other embodiments, the one or more rinse regions/sub-regions may further comprise phosphate buffered water, phosphate buffered saline, or Tris-buffered water.

In various embodiments, the binding regions/sub-regions and/or detecting regions/sub-regions comprise an aqueous medium. The binding regions/sub-regions and/or detecting regions/sub-regions comprise water. In various other embodiments, the binding regions/sub-regions and/or detecting regions/sub-regions may further comprise phosphate buffered water, phosphate buffered saline, or Tris-buffered water.

As shown in FIG. 3, the binding region/sub-region may comprise the plurality of magnetic beads 11 that have a plurality of first capture molecules 12 attached thereto, where the first capture molecules have a specific affinity for the analyte 10. Further, the binding region/sub-region may comprise a plurality of second capture molecules 13 having a detectable label 14 attached thereto, where the second capture molecules have a specific affinity for the analyte 10. The first capture molecules 12 and second capture molecules 13 may bind to different sections (e.g., epitopes) of the analyte 10 of the sample 100. Rinse sub-regions may be utilized to wash the sample.

As shown in FIGS. 1 and 3, in various examples, the detecting region 8 may comprise a molecule to initiate detection. For example, the chemical reaction that yields a product that may be detected via spectrophotometry. For example, if the second capture molecule 13 comprises horseradish peroxidase (HRP) as the detectable label 14, the detecting region 8 may comprise a detection composition 18, which comprises one or more detection molecules (e.g., Amplex Red, Luminol, or 3,3′,5,5′-Tetramethylbenzidine (TMB) 18a) and hydrogen peroxide 18b. In various examples, there is an interaction (e.g., chemical reaction) with the detectable label 14 on the second capture molecule 13 and the constituents of the detection composition 18. This interaction may be a chemical reaction. The chemical reaction may yield a fluorescent product 19. For example, when Amplex Red is utilized, the detectable label 14 may be HRP. In the presence of hydrogen peroxide 18b and Amplex Red 18a, HRP catalyzes the formation of a molecule that may be detected via fluorescence spectroscopy. It may be appreciated that the detection molecule 18a is not limited to Amplex Red, and may comprise other detection molecules, such as Luminol and 3,3′,5,5′-Tetramethylbenzidine (TMB).

As shown in FIGS. 1 and 3, a magnetic field is used to move the complexes 16 through the various regions utilized in the method. The magnetic field may be generated by a magnet (e.g., an external magnet), such as, for example, a neodymium magnet. In various other examples, the magnetic field is generated by a wire through which a current is applied. In various embodiments, an external magnet 20 is moved at a rate of 0.2 mm/s to 10 mm/s in the direction from the binding region 6 to the detecting region 8. In various other embodiments, the magnet is stationary and is positioned such that an application of a magnetic field in the absence of moving the magnet induces movement of the complexes 16 through the various regions utilized in the method. In various other embodiments, the magnet is stationary and a testing assembly in which the method is occurring is moved relative to the magnet. In various embodiments, the cartridge may move, the magnet may move, or both may move relative to each other to effect the movement of the magnetic beads along the sequential path. Without intended to be bound by any particular theory, it is considered that a magnet or device capable of generating a magnetic field with a pull force of 5-40 pounds and/or a strength of 10⁻² to 10 tesla.

As shown in FIG. 3, in various embodiments, each region described in the method is separated by an aliphatic partition 15. Sample 100 comprising the analyte 10 is contacted with the components in the binding region 6, and the analyte 10 binds to the first capture molecule 12. The solid aliphatic partitions 15a are heated to a temperature from 40° C. to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.), to melt the aliphatic partitions into liquefied partitions 15b. The analyte 10 bound to first capture molecules 12 attached to the magnetic beads 11, which are moved through the permeable liquefied aliphatic partitions 15b via application of a magnetic field. The analyte may then optionally pass through a rinse sub-region 6c or 8a following moving through a first liquefied aliphatic partition 15b. The analyte 10 is isolated from the sample 100 (e.g., whole blood) into a separate second sub-region 6b comprising the plurality of second capture molecules 13 having a detectable label 14 attached thereto. The second capture molecules 13 bind to the captured analyte 10 to form complexes 16 that comprise the analyte 10 bound to the first capture molecule 12 on the magnetic bead 11 and the second capture molecule 13 having the detectable label 14 attached thereto. The detectable label 14 remains bound to the second capture molecule 13 in this complex 16. Complex 16 may then optionally be moved through a rinsing sub-region 6c or 8a. Finally, the complexes 16 are moved into the detecting region comprising a detection composition 18, which comprises hydrogen peroxide 18b and Amplex Red 18a. The interaction (e.g., chemical reaction) between the detectable label 14, which is HRP and the detection composition 18 yields a fluorescent product 19 that may be detected by a fluorescent signal that is proportional (e.g., directly proportional) to the concentration of the analyte 10.

As shown in FIG. 1, in various embodiments, sample 100 comprising the analyte 10 is contacted with the components in the binding region 6, and the analyte 10 binds to the first capture molecule 12 attached to the magnetic bead 11 and the plurality of second capture molecules 13 having a detectable label 14 attached thereto. The second capture molecules 13 bind to the captured analyte 10 to form complexes 16 comprising of the analyte 10 bound to the first capture molecule 12 attached to the magnetic bead 11 and the second capture molecule 13. The solid aliphatic partitions 15a are heated to a temperature from 40° C. to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.), to melt the aliphatic partitions 15 into liquefied aliphatic partitions 15b. The magnetic beads 11 bound to the analyte 10 are moved through the liquefied aliphatic partitions 15b via application of a magnetic field. The complex 16 is optionally moved through a rinse sub-region 6c or 8a and through a liquefied aliphatic partition 15b. Complex 16 is moved into the detecting region 8. The detecting region 8 may comprise a detection composition 18 that comprises hydrogen peroxide 18b and Amplex Red 18a. The chemical reaction between the detectable label 14, which is HRP and the detection composition 18 yields a fluorescent product 19 that may be detected by a fluorescent signal that is proportional to the concentration of the analyte 10.

In an embodiment, various analytes may be detected using a method of the present disclosure. For example, the analyte may be an antigen, an antibody, a viral particle, or the like. For example, the antigen may be a pathogenic antigen (e.g., microbial antigen, bacterial antigen, or viral antigen). Non-limiting examples of viruses from which the antigen, antibody, or viral particle are associated include SARS-CoV-2, Hepatitis C, Epstein-Barr, Zika, Ebola, Herpes simplex, Norovirus, Influenza, Chikungunya, or the like. In various examples, the analyte is associated with SARS-CoV-2. For example, the analyte is a SARS-CoV-2 antibody.

The method may utilize various capture molecules. For example, the capture molecules comprise antibodies having a specific affinity for the analyte. The antibodies of the first capture molecule 12 and the second capture molecule 13 may be different and bind to different epitopes of the analyte 10.

The first capture molecule 12 has a magnetic bead 11 attached thereto. In various embodiments, the first capture molecule is streptavidin. The magnetic bead 11 may comprise iron oxide and be superparamagnetic. The magnetic beads 11 may have a longest linear dimension of 100 nm to 50,000 nm, including all 0.1 nm values and ranges therebetween. In various examples, the mean diameter of the magnetic beads 11 is about 1 μm. In various examples, the magnetic beads have a density of about 2 g/cm³. In various aspects, the magnetic beads 11 are coated with streptavidin.

The second capture molecule 13 has a detectable label 14 attached thereto. The second capture molecule may be The detectable label 14 may be detected directly or indirectly. For example, a detectable label 14 that is indirectly detected may catalyze or undergo a chemical reaction in the detecting region 8 with a substrate, resulting in the formation of a detectable product (e.g., detection molecule) that may be detected and optionally quantified via spectrophotometry. For example, a detectable label 14 is directly detected and optionally quantified via spectrophotometry without formation of an additional substrate or detection molecule 18a. For example, a direct detectable label is green fluorescent protein (GFP).

According to an embodiment of the present disclosure, the sample 100 may be a biological sample. Non-limiting examples of samples include whole blood, blood fractions, plasma, serum, saliva, urine, stool, sweat, mucous, tears, breast milk, semen, tissue, placental tissue, bone marrow, conditioned medium, tissue culture medium, and the like.

Various aliphatic partitions 15 may be used. For example, the aliphatic partitions 15 may comprise one or more alkanes. The alkanes may have a melting point of 40 to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.). At the melting temperature, the aliphatic partition 15 undergoes a phase change from a solid aliphatic partition 15a to a liquefied aliphatic partition 15b allowing the movement of the complexes 16 through the partition 15b. Non-limiting examples of alkanes include eicosane, docosane, hexacosane, heptacosane, nonococane, tetracosane, octadecane, and combinations thereof. In various other embodiments, the aliphatic partition 15 may be any fatty acid with a melting point of 40 to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.). In a method of the present disclosure, the aliphatic partitions 15 are initially solid 15a to prevent movement of media adjacent regions and then subsequently melted 15b to allow for movement of the analyte from region to adjacent region via application of a magnetic field.

In various embodiments, the detection composition 18 may comprise a detection molecule 18a that may be colorimetric, luminescent, or fluorescent or result in a detected molecule that may be colorimetric, luminescent, or fluorescent. In various embodiments, the detection molecule 18a may be indirectly or directly detected and optionally quantified after a chemical reaction. The detection molecule 18a or detected molecule may be detected visually or via spectrophotometry. In various examples, the detection molecule 18a and the detectable label 14 and detected molecule are the same (e.g., green fluorescent protein). In an embodiment, a colorimetric detection molecule 18a may be 3,3′,5,5′-Tetramethylbenzidine (TMB) or the like. In an embodiment, a luminescent detection molecule 18a may be Luminol or the like. In an embodiment, a fluorescent detection molecule 18a may be Amplex Red or the like. Other examples of detection molecules include, but are not limited to 2,2′-azino-bis(3-ethylbenzothiazoline-6-sulfonic acid (ABTS), 3-amino-9-ethylcarbazole (AEC), 3,3′-diaminobenzidine (DAB), and the like.

In various embodiments, the detection molecule 18a may comprise 3,3′,5,5′-Tetramethylbenzidine (TMB) and the secondary capture molecule 13 may have horseradish peroxidase (HRP) tag attached thereto as the detectable label 14. In such an embodiment, the detection solution 18 may further comprise hydrogen peroxide 18b. In the presence of hydrogen peroxide and TMB, HRP catalyzes the formation of a molecule that may produce a color change. The color change may be measured, assessed, or evaluated by an LED, an optical filter, or an optical detector, such as a photodetector or a camera.

In an embodiment, the detection molecule 18a may comprise Luminol and the secondary capture molecule 13 may have a horseradish peroxidase (HRP) tag attached thereto as the detectable label 14. In the presence of hydrogen peroxide and Luminol, HRP catalyzes the formation of a molecule that may produce luminescent product. The luminescent product may be measured, assessed, or evaluated by an optical detector, such as a photodetector or a camera. The term detection molecule may refer to the substrate that is reacted to form the detected molecule.

In an embodiment, the detection molecule 18a may comprise Amplex Red and the secondary capture molecule 13 may have a horseradish peroxidase (HRP) tag attached thereto as the detectable label 14. In the presence of hydrogen peroxide and Amplex Red, HRP catalyzes the formation of a molecule that may produce a fluorescent product 19. The fluorescent product 19 may be measured, assessed, or evaluated by an LED, an optical filter, or an optical detector, such as a photodetector or a camera.

In various embodiments, the detectable label 14 may be a fluorescent molecule that is tagged on the second capture molecule 13. The fluorescent molecule may be a fluorophore such as, but not limited to, derivatives of fluorescein, derivatives of rhodamine (TRITC), coumarin, GFP, or cyanine. The FITC-conjugated second capture molecule may produce a fluorescent product that may be measured, assessed, or evaluated by an LED, an optical filter, or an optical detector, such as a photodetector or a camera.

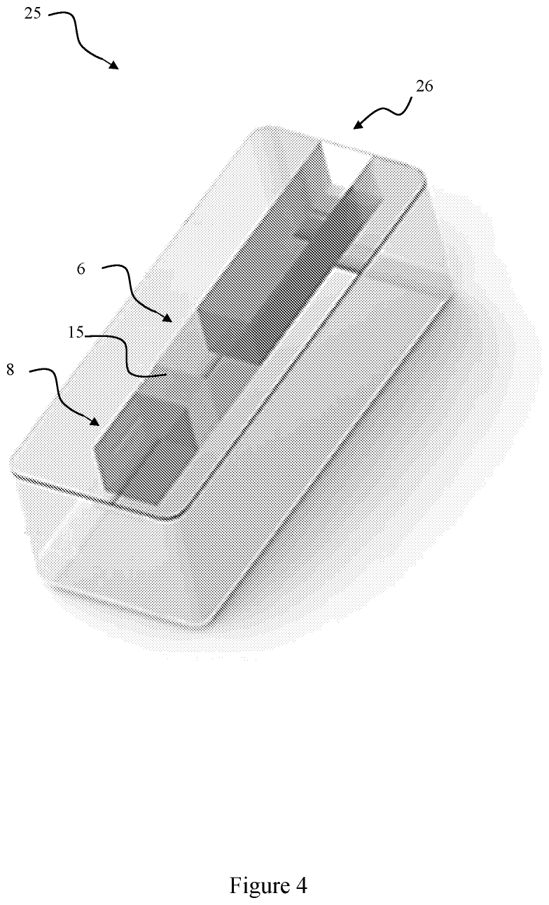

In an aspect, the present disclosure provides a testing system for detecting the presence of an analyte using the combination of thermally responsive aliphatic partitions (TRAPs) and magnetic beads (FIG. 4).

Referring to FIGS. 1, 3, and 4, the testing system may comprise a test assembly 25 and a magnet 20 or source to generate a magnetic field. FIG. 4 displays the test assembly 25. The test assembly 25 comprises an inlet 26 configured to accept a sample 100, a plurality of regions that include a binding region 6 and a detecting region 8, and at least one aliphatic partition 15 disposed therein. Adjacent regions of the plurality of regions are separated by at least one aliphatic partition 15. The binding region 6 comprises a plurality of magnetic beads 11 connected to a plurality of first capture molecules 12 and a plurality of second capture molecules 13 connected to a detectable label 14. The plurality of first capture molecules 12 are configured to bind to an analyte 10 present in a sample 100. The plurality of second capture molecules 13 are configured to bind to the analyte 10 present in the sample 100. The detecting region 8 comprises a detection composition 18 that allows spectrophotometric measurement of the analyte directly or indirectly. The magnet 20 is configured to apply a magnetic field along a sequential path of the test assembly 25. The sequential path includes the binding region 6 and the detecting region 8. The plurality of magnetic beads 11 move sequentially along the sequential path upon application of a magnetic force from the magnet 20. Thus, the magnetic beads 11 can move sequentially from the binding region 6 to the detecting region 8. The magnetic beads 11 also can move sequentially through other regions or sub-regions (such as first sub-region 6a, second sub-region 6b, binging region rinse region 6c, detecting region rinse region 8a, or detecting region 8b) along the sequential path, but in an embodiment the magnetic beads 11 move from one side of the sequential path to the other side of the sequential path.

The channel may have various shapes. For example, the channel may be cylindrical, prism-shaped, rectangular cuboid, or the like. The aforementioned shapes are merely illustrative. Other shapes are contemplated within the scope of the instant disclosure. While a uniform channel width is disclosed, other configurations using different widths, a tapering width, or a widening width are possible.

In various embodiments, a testing assembly with a 3×3 mm channel may be used. This width allows the magnetic beads 11 to pass through the aliphatic partitions 15 without breaching (meaning mixing the contents within the binding region 6 with the contents within the detecting region 8), and the layers of the assay remain separated during the melting of the aliphatic partitions 15, moving the magnetic beads 11 across liquefied aliphatic partitions 15b, and re-hardening of the aliphatic partitions 15 into solid aliphatic partitions 15a. Solid aliphatic partitions 15a melt into liquefied aliphatic partitions 15b by heating the aliphatic partitions 15 to a temperature at or between 40 to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.). The regions and sub-regions are positioned in this channel.

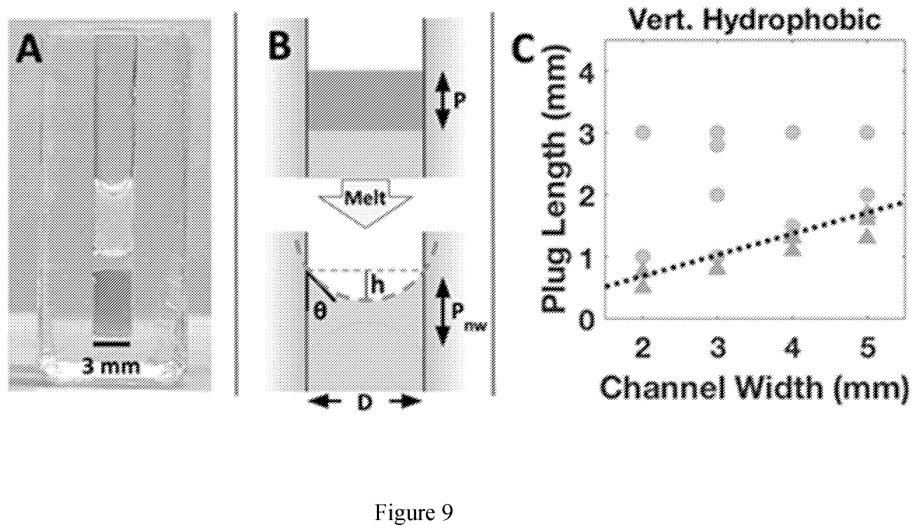

According to an embodiment of the present disclosure, at least one aliphatic partition 15, the binding region 6, and the detecting region 8 may be arranged in a horizontal channel or a vertical channel. Further, the horizontal channel or the vertical channel may be hydrophobic or hydrophilic, as shown in FIGS. 9-12, and discussed infra Example 1.

According to various embodiments of the present disclosure, the aliphatic partitions 15 may comprise one or more alkanes chosen from eicosane, docosane, hexacosane, heptacosane, nonococane, tetracosane, and octadecane. Combinations of alkanes or additional species with the alkanes are possible. The aliphatic partitions 15 may operate in at least two distinct behavior modes: (1) as a removable partition for hands-free reagent mixing following melting, and (2) as a continual partition that separates assay regions while enabling magnetic beads 11 to be pulled through following melting. The mode that the aliphatic partition 15 operates in is dependent on the geometry, position, and thickness of the aliphatic partition 15. Because of the density and polarity differences between the alkane and the aqueous reagents, the behavior is dependent upon the surface energy of the test assembly 25 and the orientation of the test assembly 25. The aliphatic partitions 15 can remain between the various regions or sub-regions and can obstruct flow of material between the regions or sub-regions except for the magnetic beads 11. The aliphatic partitions 15 can extend across an entirety of the channel in the test assembly 25 to provide this function.

According to various embodiments of the present disclosure, one aliphatic partition 15 may be configured to separate the binding region 6 and the detecting region 8. Further, the binding region 6 may comprise a plurality of sub-regions, each adjacent region or sub-region separated by one of the aliphatic partitions 15. A first binding sub-region 6a of the binding region 6 may comprise the plurality of magnetic beads 11 connected to the plurality of first capture molecules 12 having a specific affinity to the analyte 10 present in the sample 100. A second binding sub-region 6b of the binding region 6 may comprise the plurality of second capture molecules 13 connected to a detectable label 14 having a specific affinity to the analyte 10 present in the sample 100. Even further, the testing assembly 25 may comprise a rinsing sub-region 6c or 8a disposed between the aliphatic partition 15 of the first binding sub-region 6a and the aliphatic partition 15 of the second binding sub-region 6b.

According to an embodiment of the present disclosure, the magnet 20 is external to the test assembly 25, and can be moved at a rate of 0.2 mm/s to 10 mm/s along the sequential path that may comprise the binding region 6 and the detecting region 8. Thus, the magnet 20 can move along the test assembly 25 as shown in FIG. 1, 3, or 6. The magnet 20 may also be disposed on an end of the test assembly 25 in order for the magnetic field to extend across the binding region 6 and the detecting region 8 sequentially. In this configuration, the magnet 20 may be fixed or may stay localized at the end of the test assembly 25 (e.g., the short end of the testing assembly perpendicular to the movement illustrated in FIGS. 1 and 3).

According to an embodiment of the present disclosure, the detecting region 8 may comprise one or more optically transparent faces in order to allow spectrophotometric measurement of the detection composition 18 within the detecting region 8.

According to an embodiment of the present disclosure, the detection composition 18 may comprise a detection molecule 18a that may be colorimetric, luminescent, or fluorescent, and hydrogen peroxide 18b. In various embodiments, a colorimetric detection molecule may be 3,3′,5,5′-Tetramethylbenzidine (TMB). In various embodiments, a luminescent detection molecule may be Luminol. In various embodiments, a fluorescent detection molecule may be Amplex Red.

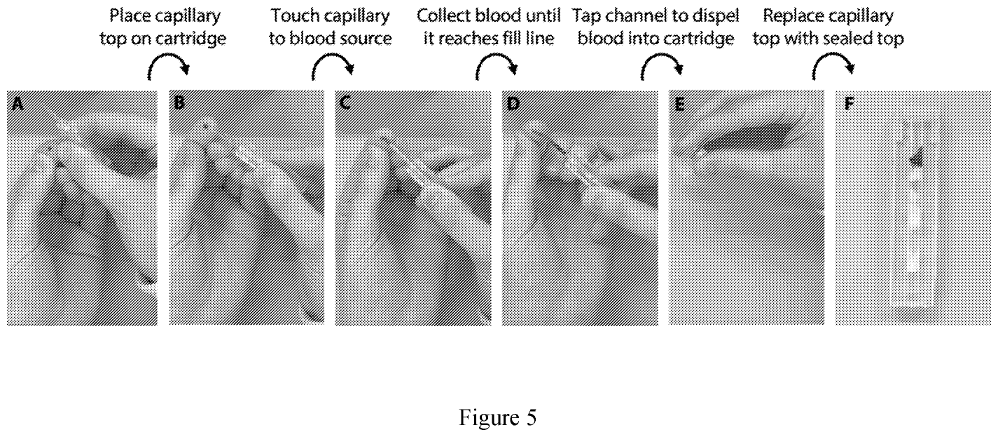

FIG. 5 demonstrates the capture of the sample 100 to be inserted into the inlet 26 of the test assembly 25. As shown in FIG. 5A, a capillary tube may prick a finger of a subject. When the capillary tube is contacted with the blood sample (FIG. 5B), the tube wicks the blood (FIG. 5C) until a precise volume is reached (FIG. 5D). The capillary tube may be inserted into the inlet 26 of the test assembly 25. The capillary tube may be tapped in order to enter the blood into the test assembly 25 (FIG. 5E). The capillary tube is exchanged with a sealed cap after the sample is loaded (FIG. 5F).

In various embodiments, one or more of the surfaces of the channel are rendered hydrophilic. They may be rendered by contacting the one or more surfaces with fetal bovine serum (FBS). Without intending to be bound by any particular theory, it is considered that the contacting increases the hydrophilicity of one or more surfaces.

This disclosure further provides a method of using the test assembly 25 for detecting the presence of an analyte 10. The capillary tube may transport the sample 100 needed into the test assembly 25 in order to perform the method 200 described herein.

The following Statements provide various examples of the present disclosure. Statement 1. A method for detecting the presence of an analyte comprising: i) contacting a sample suspected of containing the analyte together or separately with i) a plurality of magnetic beads having a plurality of first capture molecules attached thereto, wherein the first capture molecules have a specific affinity for the analyte, and ii) a plurality of second capture molecules having a detectable label attached thereto, wherein the second capture molecules have a specific affinity for the analyte, to form complexes, each complex comprising the analyte bound to the first capture molecule and the second capture molecule; ii) heating one or more solid aliphatic partitions to a temperature of 40° C. to 65° C., such that the one or more solid aliphatic partitions liquefy; iii) separating the complexes from unbound second capture molecules by selectively moving the complexes through the one or more liquefied aliphatic partitions via application of a magnetic field; and iv) detecting and optionally quantifying the signal generated from the detectable labels of the second capture molecules of the separated complexes; wherein the presence of a detectable signal is indicative of the presence of the analyte and the magnitude of the signal is indicative of the amount of the analyte in the sample.

Statement 2. A method according to Statement 1, wherein the sample is a biological sample.

Statement 3. A method according to Statement 3, wherein the biological sample is chosen from whole blood, blood fractions, plasma, serum, saliva, urine, stool, sweat, mucous, tears, breast milk, semen, tissue, placental tissue, conditioned medium, tissue culture medium, and bone marrow.

Statement 4. A method according to any one of the preceding Statements, wherein the analyte is an antibody or an antigen.

Statement 5. A method according to Statement 4, wherein the antibody is directed to a pathogenic antigen (e.g., a microbial antigen, a bacterial antigen, or a viral antigen).

Statement 6. A method according to Statement 5, wherein the viral antigen is associated with SARS-CoV-2, Hepatitis C, Epstein-Barr, Zika, Ebola, Herpes simplex, Norovirus, Influenza, or Chikungunya.

Statement 7. A method according to any one of the preceding Statements, wherein the plurality of magnetic beads has a longest linear dimension of 100 nm to 50,000 nm.

Statement 8. A method according to any one of the preceding Statements, wherein the plurality of second capture molecules have horseradish peroxidase attached thereto.

Statement 9. A method according to any one of the preceding Statements, wherein the sample is sequentially contacted with the plurality of magnetic beads having the plurality of first capture molecules attached thereto and the plurality of second capture molecules having the detectable label attached thereto.

Statement 10. A method according to Statement 9, wherein the sample is rinsed after contacting the plurality of magnetic beads having the plurality of first capture molecules attached thereto and prior to contacting the plurality of second capture molecules having the detectable label attached thereto.

Statement 11. A method according to any one of the preceding Statements, wherein the one or more aliphatic partitions comprise one or more alkanes.

Statement 12. A method according to Statement 11, wherein one or more alkanes are chosen from eicosane, docosane, hexacosane, heptacosane, nonococane, tetracosane, octadecane, and combinations thereof.

Statement 13. A method according to any one of the preceding, wherein the magnetic field is generated by a magnet.

Statement 14. A method according to any one of the preceding, wherein the detectable signal is spectrophotometric (e.g., colorimetric or fluorescent).

Statement 15. A testing system comprising: a test assembly, the test assembly comprising an inlet configured to accept a sample comprising an analyte, a plurality of regions, and at least one solid aliphatic partition disposed therein, wherein adjacent regions of the plurality of regions are separated by one of the at least one solid aliphatic partition, and the plurality of regions includes a binding region and a detecting region, and the at least one solid aliphatic partition has a melting point of 40° C. to 65° C.; wherein the binding region comprises a plurality of magnetic beads having a plurality of first capture molecules attached thereto, wherein the first capture molecules have a specific affinity for the analyte and a plurality of second capture molecules having a detectable label attached thereto, wherein the second capture molecules have a specific affinity for the analyte, wherein the detecting region comprises a detection composition configured to enable spectrophotometric measurement of the composition; and a magnet configured to apply a magnetic field along a sequential path, whereby the plurality of magnetic beads move sequentially along the sequential path upon application of a magnetic force from the magnet, wherein the sequential path includes the binding region and the detecting region.

Statement 16. A testing system according to Statement 15, wherein the analyte is an antibody or an antigen.

Statement 17. A testing system according to Statement 16, wherein the antibody or the antigen is associated with SARS-CoV-2, Hepatitis C, Epstein-Barr, Zika, Ebola, Herpes simplex, Norovirus, Influenza, or Chikungunya.

Statement 18. A testing system according to any one of Statements 15-17, wherein the at least one aliphatic partition comprises one or more alkanes.

Statement 19. A testing system according to Statement 18, wherein one or more alkanes are chosen from eicosane, docosane, hexacosane, heptacosane, nonococane, tetracosane, and octadecane.

Statement 20. A testing system according to any one of Statements 15-19, wherein the at least one aliphatic partition, the binding region, and the detecting region are arranged in a horizontal hydrophobic channel or a vertical hydrophobic channel.

Statement 21. A testing system according to any one of Statements 15-20, wherein the at least one aliphatic partition, the binding region, and the detecting region are arranged in a horizontal hydrophilic channel or a vertical hydrophilic channel.

Statement 22. A testing system according to any one of Statements 15-21, wherein the at least one aliphatic partition is configured to separate the binding region and the detecting region.

Statement 23. A testing system according to any one of Statements 15-22, wherein the binding region comprises a plurality of sub-regions, each adjacent region separated by one of the at least one aliphatic partitions.

Statement 24. The testing system according to Statement 23, wherein a first binding sub-region of the binding region comprises the plurality of magnetic beads attached to a plurality of first capture molecules, wherein the first capture molecule has a specific affinity for the analyte, and a second binding sub-region of the binding region comprises the plurality of second capture molecules attached to a detectable label, wherein the second capture molecule has a specific affinity for the analyte.

Statement 25. A testing system according to Statement 24, further comprising a rinsing sub-region disposed between the aliphatic partition of the first binding sub-region and the aliphatic partition of the second binding sub-region.

Statement 26. A testing system according to any one of Statements 15-25, wherein the magnet is external to the test assembly.

Statement 27. A testing system according to any one of Statements 15-26, wherein the magnet is configured to move across the test assembly along the sequential path.

Statement 28. A testing system according to any one of Statements 15-27, wherein the magnet is disposed on an end of the test assembly and the magnetic field extends across the binding region and the detecting region sequentially.

The following examples are provided as illustrative examples and are not intended to be restrictive in any way. These examples provide desired parameters for the method and the test system.

### Example 1

Described are the results of tests performed to determine the geometric parameters of the test assembly in a preferred embodiment. The term “TRAP” or “TRAPs” may be used interchangeably with the terms “aliphatic partition” or “thermally responsive aliphatic partitions” or “alkane partition” or “wax partitions” or “eicosane partitions.”

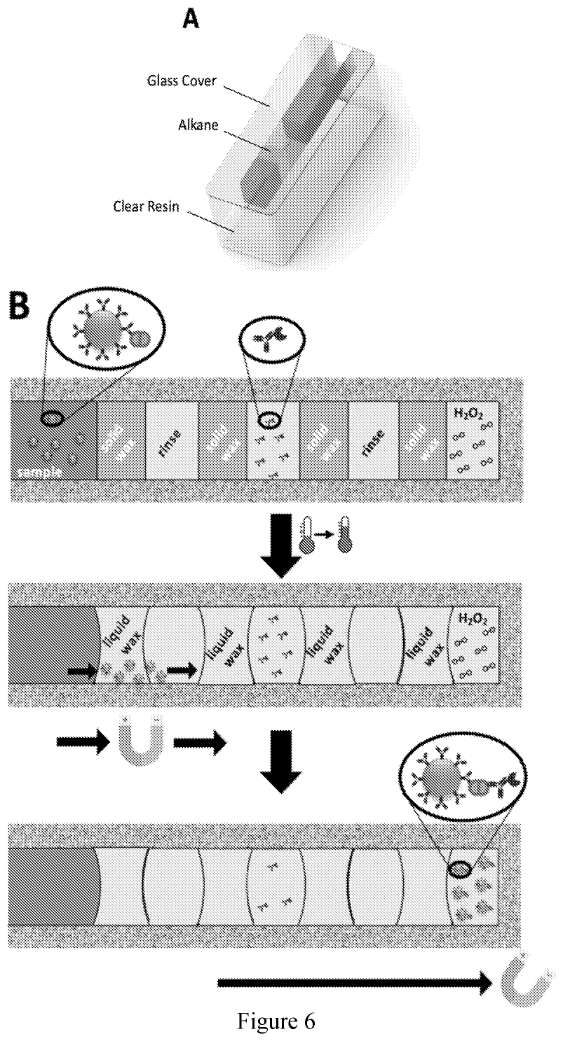

A flexible form of a TRAP in which liquefied partitions remain in place and continue to separate reagents while magnetic beads can be pulled through the liquefied partitions in a magnetofluidic assay is shown in FIG. 6. FIG. 6A depicts a 3D representation of a device with an alkane TRAP partitioning two regions comprising liquids. FIG. 6B is a schematic illustration of an immunoassay using TRAPS, in which alkane wax layers partition all reagents. When warmed, the partitions liquefy, enabling magnetic beads to be pulled from one region (e.g., reagent zone) to the next, enabling sample-to-answer assays. The alkane partitions can continue to separate reagents when confined within sufficiently small geometries. By using phase-changing alkanes as partitions, the reagents can remain partitioned even during shipping and handling, while magnetofluidic manipulation of reagents across partitions can be conducted following thermal actuation.

Described herein is the design and rules that dictate whether the partition is removed for reagent addition or remains stationary for continual partitioning. Because of the density and polarity differences between the alkane and the aqueous reagents, the behavior is dependent upon the surface energy of the reaction vessel and the vessel orientation (i.e., vertical versus horizontal). This example describes the design rules for all permutations of these conditions. The design rules for pulling magnetic microbeads through liquefied partitions without causing reagent breaches was investigated.

All of the test assemblies tested in this example were 3D-printed with resin from Formlabs. The cover slips were obtained from Fisher Scientific. The blue and yellow dyes were purchased from Wilton Color Right and were used to color water. The alkane used in the experiments described herein was n-eicosane (melting point 42° C.), 99%, purchased from Alfa Aesar. Streptavidin magnetic beads (1.05 μm diameter) were purchased from BioLabs. The glue used to adhere glass to resin was Scotch liquid super glue. Heat-inactivated fetal bovine serum (FBS) from American Type Culture Collection (ATCC) was used as a means to hydrophilize the surfaces of the test assemblies. Amplex Red was purchased from Biotium, and the hydrogen peroxide used to react with Amplex Red was from Fisher Scientific. The biotinylated rabbit IgG antibody and the HRP-conjugated anti-rabbit IgG antibody were both manufactured by ThermoFisher. The bead wash buffer was made with Tris and NaCl, both from Sigma Aldrich. Finally, the phosphate buffer was made with components from JT Baker.

Test assemblies with various channel geometries were fabricated of resin 3D printed by a Form 2 stereolithography printer (Formlabs). The parts were cleaned with isopropyl alcohol (IPA) to ensure no uncured resin remained on the printed test assembly. The outer dimensions of each test assembly were the same: 10 mm wide, 8 mm in height, and 25 mm long. There were five different channel geometries used in this example, all were 22 mm long: 2×2 mm, 3×3 mm, 4×4 mm, 4.5×4.5 mm, and 5×5 mm. One 22 mm long face of these inner dimensions was open to air when printed and was covered by a glass cover slip, 10 mm wide, 25 mm long, and 0.15 mm thick, and glued in place. Some channels were modified to be hydrophilic. These hydrophilic channels were filled with FBS and soaked at room temperature for 2 hours to increase hydrophilicity.

To quantify the hydrophobicity of the resin material in these experiments, the contact angle of a drop of water on a resin slab was measured. This was done by placing the slab and a drop of water onto a contact angle goniometer and recording the resulting angle. The contact angle of the untreated resin was found to be 82.0° (S.D.=2.8°, n=5), while the contact angle of the FBS-treated resin was measured as 38.8° (S.D.=8.3°, n=5), indicating an increase in hydrophilicity.

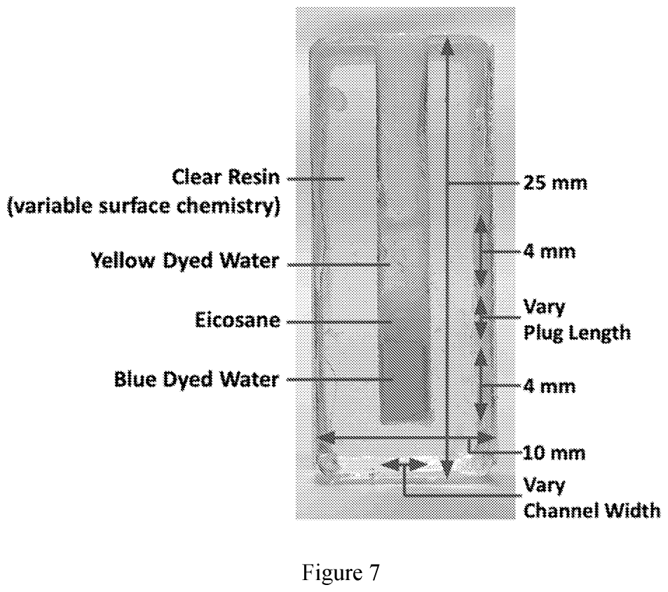

To investigate the behavior of TRAPs in various geometries, the channels were filled with two layers of water separated by a TRAP. First, channels were filled with the respective volume of blue dyed water to fill 4 mm along the channel, as shown in FIG. 7. FIG. 7 is a labeled photograph of a TRAP with yellow dyed water on top of eicosane on top of blue dyed water. To deposit eicosane, it was first melted and then pipetted on top of the water. Eicosane was melted by placing it in a glass vial on a hot plate at 120° C. Melted eicosane quickly hardened when deposited onto the water, sealing the blue dyed water layer. Yellow dyed water was pipetted on top of the hardened eicosane. The volume of yellow dyed water equaled that of the blue dyed water, except for the case of the 5×5 mm channels, for which the volume of yellow dyed water was doubled to ensure the eicosane layer never contacted air when the channel was positioned horizontally. Once filled, each test assembly was placed on a 60° C. hot plate either horizontally (resting on the 25×10 mm resin face) or vertically (resting on the 10×8 mm resin face). After about 3 minutes, the eicosane melted and was either breached, allowing the two dyes to mix, or remained in place, preventing the two dyes from mixing. A breach is defined by green color appearing in the region near the TRAP or if any part of the TRAP was detached from the channel walls. The volume of eicosane used was experimentally varied to observe which plug lengths (calculated by dividing the volume of eicosane by the channel cross-sectional area) caused a TRAP to be breached or to stay in place.

To investigate the potential to use TRAPs in magnetofluidic methods, the stability of stationary TRAPs and the leakage of TRAPs as magnetic beads are pulled through the liquefied partitions was tested. Water was placed in a 3×3 mm channel such that 4 mm of the channel was filled (36 μL). 18 μL of melted eicosane was placed on top of the water to fill another 2 mm of the channel. 36 μL of a solution containing 10 μM FAM fluorescein and g magnetic beads in water was then placed on top of the eicosane layer. The peak absorbance wavelength of FAM was 495 nm, while the peak emission wavelength was 520 nm. After the channel was set up, fluorescence measurements of both sides of the TRAP were taken. The channel was then placed on a 60° C. hot plate. Once the eicosane melted, the magnetic beads were gathered against the glass cover by holding a neodymium magnet against the glass on the outside of the channel. The magnet was then moved along the glass to the other side of the TRAP at about 2 mm/s, pulling the beads along with it. The magnet was removed and the channel was taken off the hot plate. Then, once the eicosane re-hardened, another set of fluorescence measurements of both sides of the TRAP was taken.

To study the geometry constraints to prevent leakage, blue dyed water was placed into a 3×3 mm channel such that 4 mm of the channel was filled up (36 μL). Melted eicosane was placed on top of the water. Yellow dyed water with magnetic beads filled 8 mm of the channel on top of the eicosane (72 μL). The channel was then placed on a 60° C. hot plate. Once the eicosane melted, the magnetic beads were gathered against the glass cover by holding a neodymium magnet against the glass on the outside of the channel. The magnet was then moved along the glass to the other side of the TRAP, pulling the beads along with it. The magnet was removed and the channel was taken off the hot plate. The TRAP was classified as “bridged” if color mixing was observed or if the eicosane had separated from the glass surface. The amount of eicosane and magnetic beads were experimentally varied to determine which combinations of partition thickness and bead mass caused the TRAP to bridge or remain intact.

To investigate if the magnetofluidic method detailed in the present disclosure could be implemented in an immunoassay, tests were performed to determine whether antibodies captured on streptavidin magnetic beads could be transferred across one, two, and three layers of melted TRAPs without a significant amount dissociating from the beads. 1 mg of the streptavidin-coated magnetic microbeads was incubated with 35 μg biotinylated rabbit IgG antibody in solution for 90 minutes at room temperature. They were then washed by gathering them to the side of the tube, aspirating out the supernatant, and rinsing them with 200 μL of 25 mM Tris and 150 mM NaCl buffer three times. After the final rinse, 56 μg of HRP-conjugated anti-rabbit IgG antibodies in phosphate buffer were added to the mass of beads and incubated for 10 minutes at room temperature (from 20° C. to 25° C.). Then the wash step was repeated, except after the final rinse, 60 μL of phosphate buffer was added to the mass of beads. 1 mg of streptavidin-coated magnetic microbeads was rinsed three times with 200 μL of 25 mM Tris and 150 mM NaCl buffer and put into 60 μL of phosphate buffer. 56.4 μL of this batch of beads were added to 0.6 μL of the antibody-bound beads for a 1:100 dilution of the antibody-bound beads. A 30 μL solution of 0.05 mM Amplex Red and 1 mM hydrogen peroxide was added to the bottom of a 3×3×42 mm channel. Then, 30 μL of melted eicosane were placed on top of the solution. Three sets of three channels were designated to represent three different scenarios: magnetic beads travelling through one, two, and three layers of melted TRAPs. In the first set, 26 μL of phosphate buffer and 4 μL of the prepared 1:100 antibody-bound magnetic bead solution were placed on top of the eicosane layer. In the second set, a 50 μL phosphate buffer rinse layer was placed on top of the eicosane, followed by 30 μL of melted eicosane. Then 26 μL of phosphate buffer and 4 μL of the prepared 1:100 antibody-bound magnetic bead solution were added. In the third set, a 50 μL phosphate buffer rinse layer was placed on top of the eicosane followed by 30 μL of melted eicosane, then 50 μL of phosphate buffer, then 30 μL of melted eicosane. Finally, 26 μL of phosphate buffer and 4 μL of the prepared 1:100 antibody-bound magnetic bead solution were added. In each case, the test assemblies were placed on a 60° C. hot plate to melt the eicosane layers. Once the eicosane was melted, the magnetic beads were gathered against the glass cover by holding a neodymium magnet against the glass on the outside of the channel. The magnet was moved along the glass at a rate from 0.2 mm/s to 10 mm/s to the Amplex Red and hydrogen peroxide layer, pulling the beads along with it. The magnet was removed and the channel was taken off the hot plate. 10 minutes later, a fluorescence measurement of the final layer was taken. As a no-TRAP control, three additional channels were prepared where 4 μL of the prepared 1:100 antibody-bound magnetic bead solution were placed at the bottom of the channels, a magnet was applied to pull the beads to a side of the channels, the supernatant was aspirated out, and 30 μL of the 0.05 mM Amplex Red and 1 mM hydrogen peroxide solution were added to the beads. 10 minutes later, a fluorescence measurement of the reaction was taken.

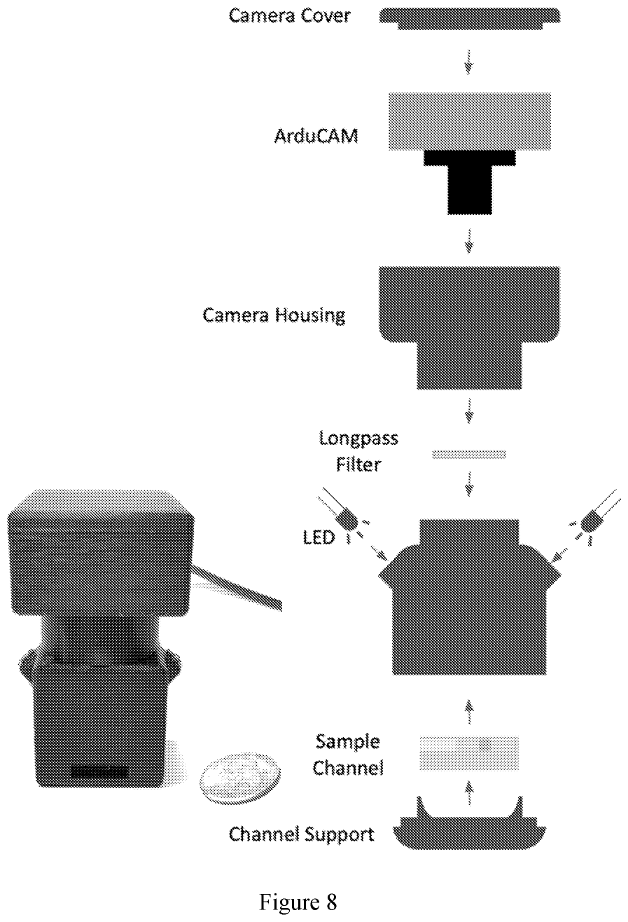

All fluorescence measurements were taken with a florescence plate reader, and the results are shown in FIGS. 9-12. All fluorescence measurements were taken by placing a device into a portable fluorescence reader as shown in FIG. 8. The device comprises a 3D printed support that houses an ArduCAM MT9M001 Camera with an ArduCAM USB2 Camera Shield (ArduCAM), a longpass filter (Thorlabs) on the lens of the camera (530 nm for the FAM fluorescein readings or 570 nm for the Amplex Red readings), two LEDs to excite the sample (470 nm blue for the FAM fluorescein readings or 525 nm green for the Amplex Red readings), and a support that secures the channels in a precise location relative to the camera.

The observations using alkane to initially separate assay reagents in microtubes showed that wax liquefaction led to partition breach in which reagents could automatically be added and mixed on-demand. In a narrower channel, surface tension and the hydrophobic interactions between wax and resin could keep the liquefied alkane partition in place despite the density difference between it and the surrounding liquids.

In this example, geometric design rules were determined, mathematically and experimentally, that govern when the liquefied alkane barrier continues to partition and when it breaches, promoting reagent mixing.

FIG. 9 shows the results of a vertically oriented channel with a hydrophobic channel wall. FIG. 9A displays a photograph of a melted TRAP in a vertical hydrophobic channel. FIG. 9B displays a 2-dimensional schematic of a cross-section along the center of the channel parallel to the image plane in FIG. 9A before and after melting the TRAP. FIG. 9C displays data representing the TRAPs that remained intact (circle) and TRAPs that breached (triangle), as well as the threshold (dashed line) determined by a mathematical approximation.

It was observed that because of the hydrophobic surface, the liquefied eicosane formed a concave meniscus at each aqueous interface as shown in FIG. 9B. The angle of the water-eicosane boundary against the wall at a corner was measured to be 45° with a standard deviation of 5.9°. Because of the meniscus shapes, it was concluded that as the partition thickness (P) decreases, the thickness of the partition at the center point will reach zero for a non-zero partition thickness, thus causing a breach in the eicosane plug and allowing the two initially separated layers to mix.

To mathematically predict the threshold partition length (Pₜₕ) that determines whether a breach will occur, the following assumptions were made: (1) the meniscus at a water-eicosane interface is a portion of the surface of a sphere constrained by the square cross-section of a channel whose centerline includes the center of the sphere (see dashed curve in FIG. 9B); and (2) breaching occurs (e.g., only occurs) when the two meniscuses touch along the centerline of the channel (i.e. when Pₙ−2h=0, where Pₙ is the distance between the two meniscuses at a corner of the channel and h is the depth of a meniscus at the centerline of the channel depicted in FIG. 9B).

Equation 1 dictates how the threshold value of the partition thickness Pₜₕ varies with respect to the channel width D and contact angle θ, which is the angle between the eicosane and water at a corner of the channel (FIG. 9B). It is important to note that the cross-section schematic shown in FIG. 9B is parallel to the image plane in FIG. 9A. Because the angle calculations for θ were done at the corners of the channel, Equation 1 operates along the diagonal of the channel, and thus √2 D is used.

$${P}_{t h} = \sqrt{2} D \frac{1 - sin ( θ )}{cos ( θ )} - \frac{π}{6 \sqrt{2} D} \times \frac{1 - sin ( θ )}{cos ( θ )} [ {( \frac{3}{4} \sqrt{2} D )}^{2} + {( \frac{1}{2} \sqrt{2} D \frac{1 - sin ( θ )}{cos ( θ )} )}^{2} ] \qquad ( 1 )$$

The data from the experiment confirmed the predictive ability of the mathematical derivation. As shown in FIG. 9C, when an eicosane partition in the channel has a partition thickness below the estimated threshold, the melted eicosane layer breaches and allows the liquids to mix (FIG. 9C triangles). When the partition thickness is above the estimated threshold, the melted eicosane remains intact and no mixing occurs (FIG. 9C circles). Because the TRAPs remain intact at relatively small partition thicknesses, the vertical hydrophobic channel appears to be suitable for applications for partitioning reagents in sample-to-answer magnetofluidic immunoassays.

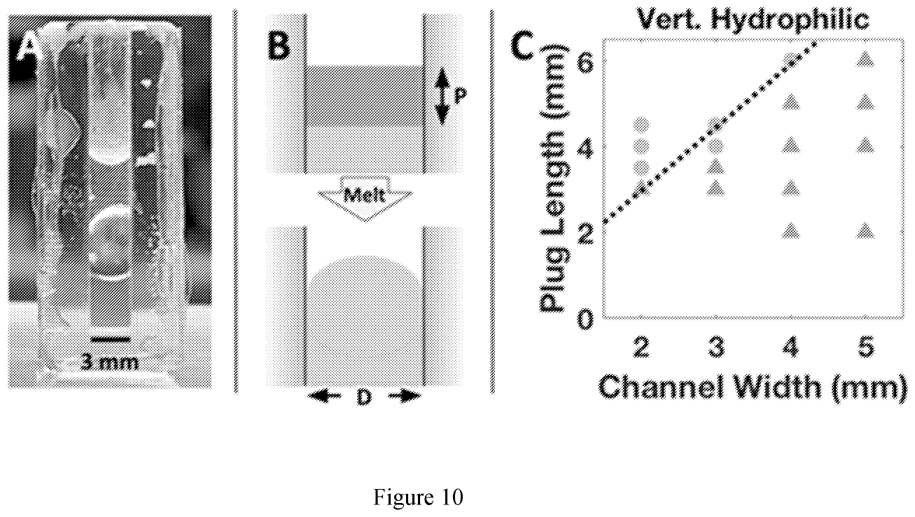

FIG. 10 shows the results of a vertically oriented channel with a hydrophilic channel wall. FIG. 10A displays a photograph of a melted TRAP in a vertical hydrophilic channel. FIG. 10B displays a 2-dimensional schematic of a cross-section along the center of the channel parallel to the image plane in FIG. 10A before and after melting the TRAP. FIG. 10C displays data representing the TRAPs that remained intact (circle) and TRAPs that breached (triangle), as well as the threshold (dashed line) determined by a mathematical approximation.

It was demonstrated that TRAPs can maintain partitioning capability in a vertical channel despite gravity, due to surface tension and the hydrophobic interactions between wax and resin. Next, the behavior of the system when the channel has a hydrophilic wall surface was investigated. In this case, the hydrophilic wall caused liquid wax to separate off of the wall, especially at thinner partition thicknesses (FIG. 10). This enabled breaching along the corners of the channel and ultimately led to floating wax in many cases.

To mathematically predict the threshold that determines whether a breach will occur given a certain partition thicknesses and square channel width, the following assumptions were made: (1) the wax will form a sphere and float up through the water with no interference from channel walls; and (2) with interference from channel walls, the wax will remain (e.g., only remain) in place as a partition when the radius of the sphere it would become given the input volume equals the length from the center of the channel to a corner. Equation 2, which determines the threshold partition length at which a TRAP will breach, is derived by equating the threshold input volume (D×D×Pₜₕ) with the assumed spherical shape that forms when the wax melts and attempts to break free of the channel walls. The threshold Pₜₕ is plotted as a dashed black line in FIG. 9C.

The experiments confirm the predictive capability of this equation. Most combinations of partition thickness and channel width that were below the threshold line resulted in the mixing of the two initially partitioned liquids (FIG. 10C triangle). Likewise, most combinations of partition thickness and channel width that were above the threshold line resulted in a stationary partition that prevented the two liquids from mixing. Given that the hydrophilic case results in breaching for even moderately thick partitions, this arrangement appears to be suitable for applications in which the partition is to be removed in order to automate precise reagent additions and mixing.

$${P}_{t h} = \frac{\sqrt{2}}{3} π D \qquad ( 2 )$$

FIGS. 9 and 10 demonstrate the impact of surface energy on the behavior of the liquefied partition. Because gravity also plays an important role in the behavior of the liquefied partitions, the above investigations were repeated for a channel placed horizontally.

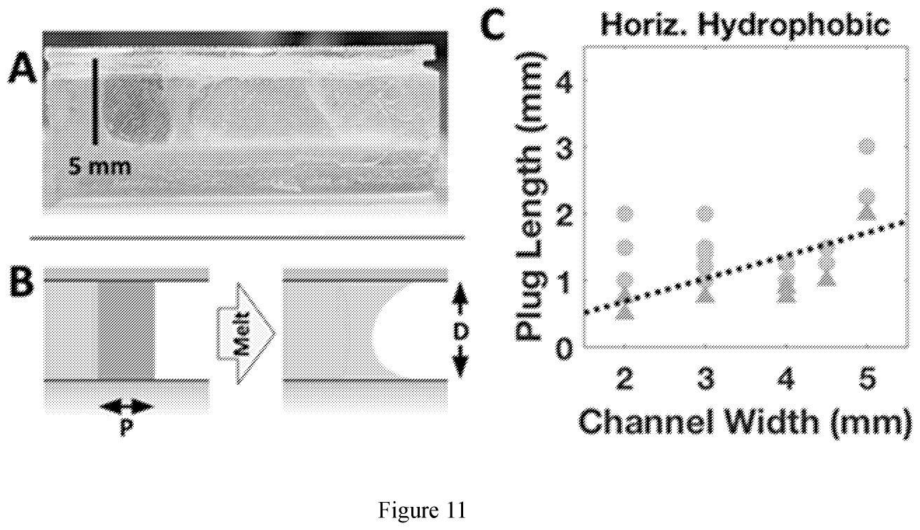

FIG. 11 shows the results of a horizontally oriented channel with a hydrophobic channel wall. FIG. 11A displays a photograph of a melted TRAP in a horizontal hydrophilic channel. FIG. 11B displays a 2-dimensional schematic of a cross-section along the center of the channel parallel to the image plane in FIG. 11A before and after melting the TRAP. FIG. 11C displays data representing the TRAPs that remained intact (circle) and TRAPs that breached (triangle), as well as the threshold (dashed line) determined by a mathematical approximation.

As shown in FIG. 11, the meniscuses observed in the vertical hydrophobic case were present again. However, under the influence of gravity, these meniscuses were asymmetric, with more wax along the top wall of the channel, especially in larger channel widths (FIGS. 11A and 11B). Thus, it can be concluded that when the partition thickness is thin enough, the breach will still occur near the center of the channel. Because the breaching behavior was similar to the vertical hydrophobic case and since the asymmetry due to gravity is not as prevalent in smaller channel widths, the same mathematical analysis as in the vertical hydrophobic case was used (Equation 1).

As expected, this model matched with the data at smaller channel widths but is less accurate when predicting the behavior at larger channel widths (>3 mm, FIG. 11C). The experimental results demonstrate that the horizontally oriented hydrophobic channel may be a viable system for continually partitioning reagents in a magnetofluidic assay.

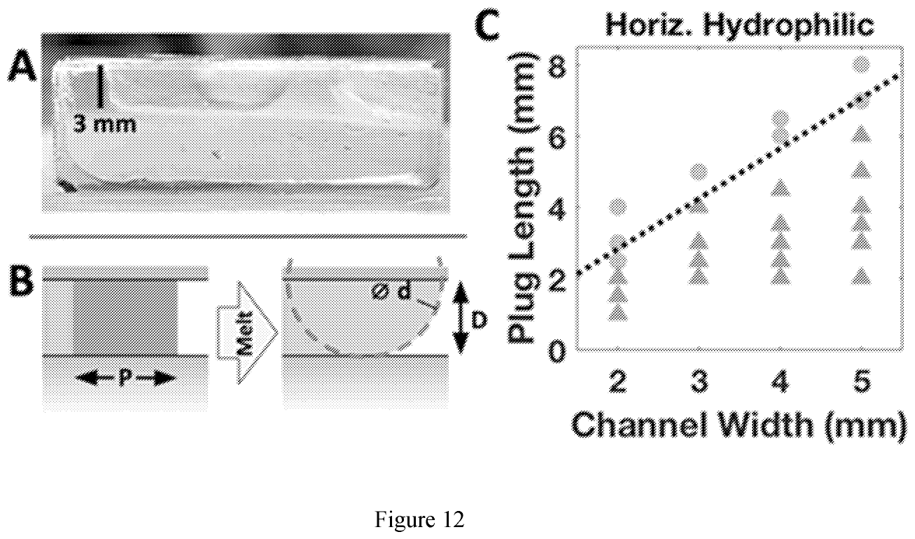

FIG. 12 shows the results of a horizontally oriented channel with a hydrophilic channel wall. FIG. 12A displays a photograph of a melted TRAP in a horizontal hydrophilic channel. FIG. 12B displays a 2-dimensional schematic of a cross-section along the center of the channel parallel to the image plane in FIG. 12A before and after melting the TRAP. FIG. 12C displays data representing the TRAPs that remained intact (circle) and TRAPs that breached (triangle), as well as the threshold (dashed line) determined by a mathematical approximation.

The horizontal hydrophilic channels resulted in similar wax behavior as the vertical hydrophilic case in which the wax tended to detach from the channel walls and form a spherical shape when subjected to no external interference. However, because in that situation gravity was in a perpendicular direction relative to the length of the channels, the sphere of wax floated up against the surface of the glass cover, which resulted in the formation of the bulged shape shown in FIG. 12A. It was observed that the wax sealed the channel when this bulged shape pressed against the side walls of the channel such that the wax reached all corners of the channel. At this point, the wax in contact with a side wall formed a near-semicircular shape. Because of this, the mathematical analysis estimated the volume of the wax at the threshold as the area of a semicircle (diameter d equals the channel width D) times the channel width with an adjustment factor, a, which modifies the equation to account for deviations in the cross-section from a perfect semicircle across the width of a channel. The empirically determined term a is necessary because of the combinations of partition thicknesses and channel widths that result near the threshold of a breached TRAP. In this domain, the force of the eicosane surface tension cause a liquid bridge to form that extends the eicosane semicircle shape towards the wall to seal the channel, though the geometry would result in a semicircle with a radius smaller than the width of the channel. Equating this formula to the volume of the input wax (D×D×P) and setting α=0.9 (determined empirically) results in Equation 3, plotted as a dashed line in FIG. 12C. The data matches to this mathematical approximation, and each TRAP above the threshold line remained intact (FIG. 12C circles) and most points below the line allowed the liquids to mix (FIG. 12C triangles).

As with the vertical hydrophilic channels, the experimental results demonstrate that the horizontal hydrophobic channels result in relatively unstable partitions. Thus, this arrangement may be viable for applications in which the partition must be removed in order to automate precise reagent additions and mixing.

$${P}_{t h} = α \frac{π}{2} D \qquad ( 3 )$$

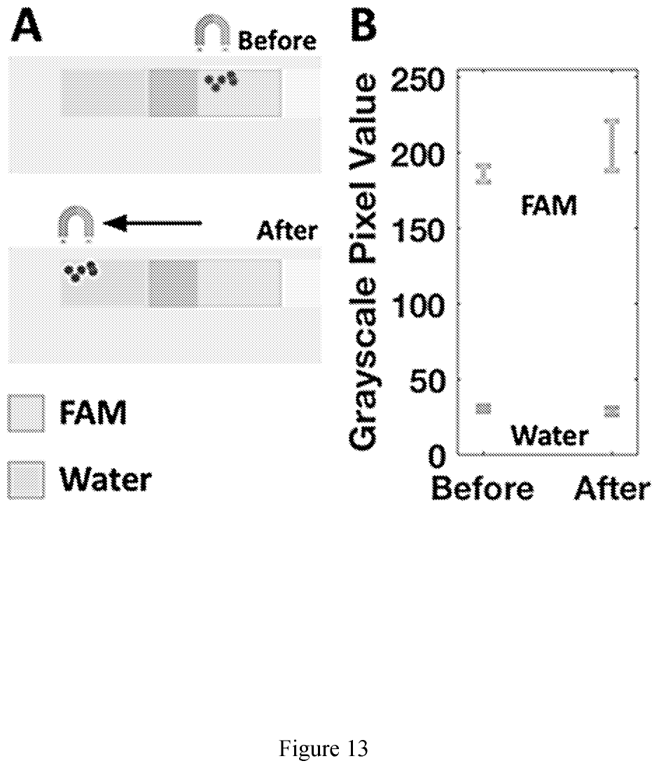

The experiments above (FIGS. 9 and 11) demonstrate that when confined in small hydrophobic channels, the TRAPs remain stationary upon liquefaction. It was hypothesized that the liquefied partitions would be permeable to magnetic beads driven by an external magnet and would remain stationary as the beads are pulled through, thus continuing to partition the solutions while the beads are transferred from one solution to the other. To verify this capability, a channel in which a TRAP partitioned water and an aqueous solution with the fluorophore FAM was tested. The magnetic beads were also loaded into the FAM solution. A fluorescence reader (shown in FIG. 8) was used to measure the fluorescence on both sides of the partition before and after the magnetic beads were pulled through the TRAP. As shown in FIG. 13, the beads were pulled from one side of the partition to the other without disturbing the partition. The beads were pulled using an external magnet moved from 0.2 mm/s to 10 mm/s. Furthermore, no fluorescence change was measurable on the destination side of the partition, suggesting that the TRAP continued to partition during the bead transfer.

Specifically, FIG. 13A is a schematic depicting a fluorescence measurement taken before and after magnetic beads were transferred across a TRAP. FIG. 13B displays data showing fluorescence values of the FAM side (green) and water side (blue) of the TRAP.

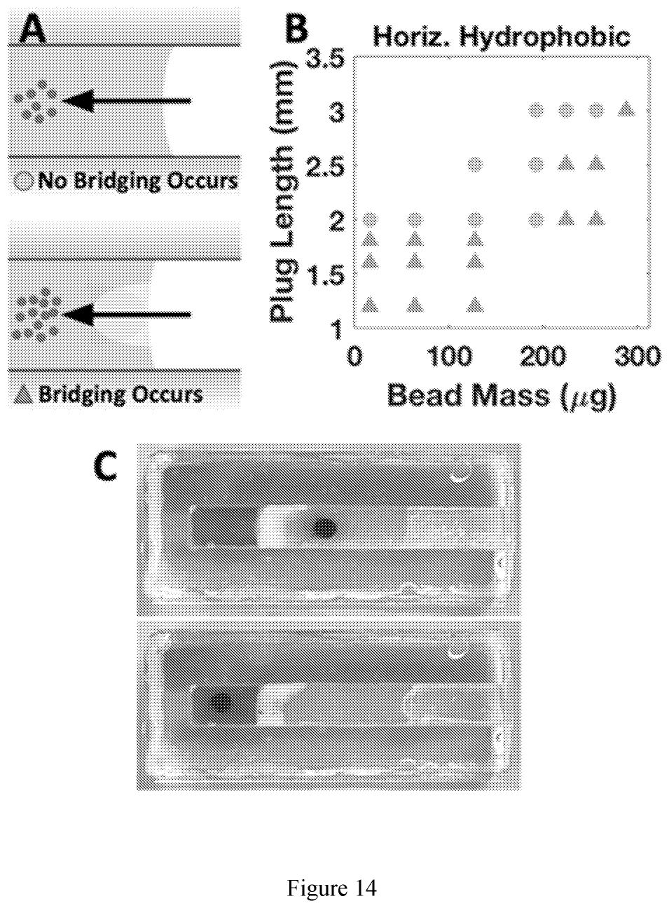

While the system in FIG. 13 shows continued partitioning, it was hypothesized that if the partition length was short enough and if the bead cluster volume was large enough, bridging between the partitioned zones could occur (FIG. 14A). Thus, the geometric design rules must also account for this possibility. To investigate this, the partition thickness and the bead volume was varied, and it was determined which conditions resulted in breaching of the partition. The data in FIG. 14 demonstrates that thinner partitions and larger quantities of beads led to bridging (triangles). Bridging occurs when the cluster of beads that is pulled across a TRAP connects the two liquids on either side. When this occurs, the TRAP does not reseal once the two aqueous layers are bridged.

Specifically, FIG. 14A displays a schematic depicting that fewer beads and a thicker TRAP (top of figure) will not bridge, while more beads and a thinner TRAP (bottom of figure) may bridge, causing the two liquids to breach. FIG. 14B displays data showing which experimental combinations of plug length and bead mass caused the TRAPs to remain intact (circles) or bridge (triangle). FIG. 14C displays a photograph of 64 μg of magnetic beads traveling through a 2 mm TRAP plug length with no TRAP bridging. The top of FIG. 14C shows the magnetic beads before movement, and the bottom of FIG. 14C shows the magnetic beads after movement via a magnetic field.

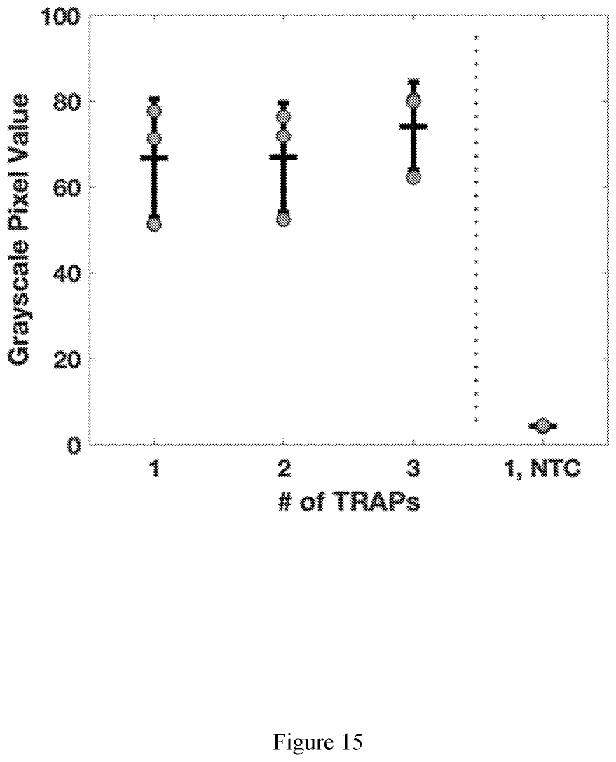

FIG. 15 displays the fluorescence signals of the antibody-bound beads that traveled through one, two, or three TRAPs, and a no target control (NTC) that included beads with no antibodies that traveled though one TRAP (n=3). It was hypothesized that magnetic beads functionalized with proteins that can capture biomarkers are capable of transferring biomarkers across liquefied TRAPs. This step is necessary in common immunoassays in which biomarkers or labeling antibodies are bound in one step and then transferred to a rinse step and subsequent binding steps. Because the temperature and hydrophobicity of the wax could cause antibodies and antigens to denature, the binding stability was assessed as the antibody complexes were carried through a TRAP via a magnetic bead. HRP-conjugated anti-rabbit IgG antibodies were used as a model antigen (i.e., captured biomarker), captured by rabbit IgG. The antibodies attached to the magnetic beads to form a sandwich. The bound complex was transferred across different numbers of TRAPs and loss of binding was quantified (using Amplex Red and hydrogen peroxide on the destination side of the TRAP). The data in FIG. 15 demonstrates no significant difference between fluorescence signals of the antibody-bound beads after travelling through a single TRAP and through subsequent numbers of TRAPs. Since a substantial amount of signal is seen after the antibody-bound beads are transferred (compared to the no target control), it was concluded that the TRAPs are compatible with immunoassays.

### Example 2

To maximize access to SARS-CoV-2 serological (antibody) tests, point-of-care (PoC) options are used. PoC tests require sample-to-answer functionality, which is challenging with whole blood. This example demonstrates a sample-to-answer SARS-CoV-2 antibody test from whole blood using automated thermally actuated valves. Higher-order alkanes serve as partitions between immunoassay regions (e.g., zones (sample/bind, rinse, detection)); upon warming, the partitions liquefy, enabling magnetic beads to be moved through each zone while continuing to partition the reagents. The instant data show a detection limit of 0.7 ng/mL SARS-CoV-2 antibodies, multiple orders of magnitude lower than clinically relevant concentrations.

This example displays a SARS-CoV-2 serological test from whole blood that eliminates precise manual steps without the need for equipment. As shown in FIG. 1, assay reagent regions are initially partitioned using eicosane, a solid wax at ambient temperature; when warmed, the wax liquefies. As depicted in Example 1, it was found that when confined in a narrow channel, the liquefied partitions remain in place, but allow magnetic beads to be pulled from one region to another by an external magnet, enabling the wax to be used as permeable partitions. In this sample-to-answer assay, beads functionalized with SARS-CoV-2 spike protein capture patient antibodies from the blood sample (along with a labeling antibody to complete the sandwich assay) and are then pulled through liquefied partitions, first into a rinse region, then into a detecting region.

Described are the results of SARS-CoV-2 antibody detection utilizing the thermally responsive aliphatic partition and magnetic bead combination, as described in the present disclosure. This testing method separated reagent compositions into three regions/sub-regions, a binding region, a rinse region, and a detection region by alkane partitions made of eicosane wax. These alkane partitions continued to separate reagent compositions in the binding region, the rinse region, and the detection region while in a solid state at ambient temperature and while in a liquefied state after heating the alkene partitions to 42° C.

As shown in FIG. 1, the binding region housed magnetic beads functionalized with SARS-CoV-2 spike proteins and horseradish peroxidase labeled (HRP-labeled) antibodies. When a whole blood sample was added to this zone, SARS-CoV-2 antibodies present in the sample bound to the magnetic beads, while HRP-antibodies in solution labeled the SARS-CoV-2 antibodies to form a sandwich.

After a 30-minute incubation period, the alkane partitions were warmed to 42° C., causing the alkane partitions to become liquefied. Once liquefied, an external magnet was moved in the direction from the binding compartment to the rinsing compartment. The movement of the external magnet in this direction pulled the magnetic beads with the antibody sandwiches into the rinse compartment to rinse the bound sandwiches from unbound magnetic beads and unbound HRP-labeled antibodies.

The external magnet was then moved in the direction from the rinse region to the detecting region. The movement of the external magnet in this direction pulled the magnetic beads with the antibody sandwiches into the detecting region containing Amplex Red and H₂O₂. The RP-labeled antibody within the sandwich reacted with the Amplex Red and the H₂O₂, to convert Amplex Red into a fluorescent product. The detectable fluorescence was proportional to the concentration of SARS-CoV-2 antibodies, quantifiable in a custom portable reader that contains an integrated nichrome heater, an LED, and an Arducam camera.

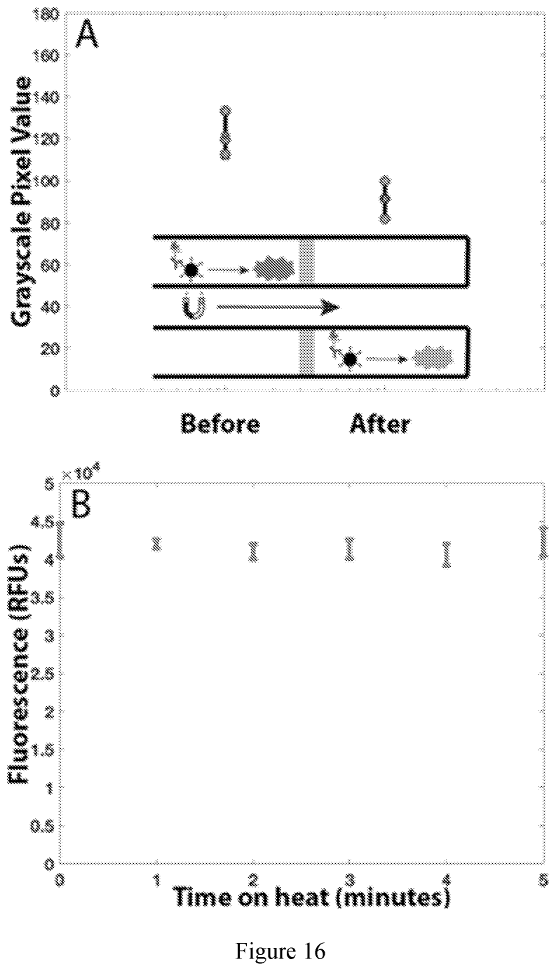

In this example, the impact of both the eicosane wax and heat on the stability of the antibody sandwiches was assessed. To do this, antibody sandwiches bound to the magnetic beads were pulled across one alkane partition and into a second region containing Amplex Red and H₂O₂, via a magnetic field of an external magnet. The results are shown in FIG. 16A. As shown in this figure, the resulting fluorescence in the detection zone was 76% of the total fluorescence produced from the bead sandwiches initially added, suggesting that the majority of the labeling antibodies remain bound to the beads and crossed the alkane partition. Additionally, the stability of the antibody sandwiches when exposed to 60° C. was tested, as shown in FIG. 16B. This temperature is significantly higher than the melting temperature of the eicosane. To do this, a sandwich was formed with beads functionalized with spike protein, an anti-spike-protein antibody, and an HRP-labeled secondary antibody. The beads were heated in solution, and then the amount of HRP bound to the beads was assessed using Amplex Red. There was no significant loss of bound antibodies in 5 minutes, demonstrating that the warming step to melt the eicosane will not negatively affect the assay performance.

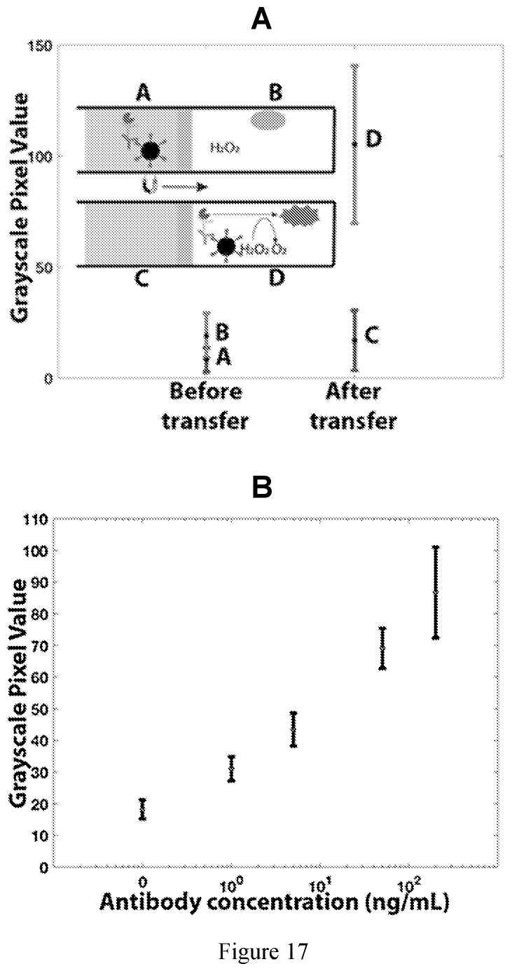

Further, in this example, the critical step of binding the antibodies from whole blood, along with a labeling secondary antibody, and removing them while keeping the blood separate was assessed. FIG. 17A demonstrates that the spike-functionalized magnetic beads were able to capture SARS-CoV-2 antibodies (spiked into commercially purchased human blood) and HRP-labeled antibodies that could be pulled into a detecting region with Amplex Red via the magnetic field of an external magnet. No visible blood was observed crossing into the detecting region.

The concentration detection of SARS-CoV-2 antibodies was assessed. As shown in FIG. 17B, in this example, the detection limit was 0.7 ng/mL, which is about 100 times lower than patient samples though previously tested positive for SARS-CoV-2.

This example demonstrates a sample-to-answer method for detection of SARS-CoV-2 antibodies with a limit of detection of 0.7 ng/mL. The detection limit is also comparable to other proposed PoC systems for SARS-CoV-2 antibody detection, yet does not require external or manual blood preparation.

This method for serological detection of SARS-CoV-2 uses alkane partitions to integrate blood preparation steps into a PoC platform for true sample-to-answer diagnosis. Also demonstrated was the detection of SARS-CoV-2 antibodies from whole blood at concentrations well below physiological relevance without the need for precise manual steps or equipment.

### Example 3

Described are the results of SARS-CoV-2 antibody detection utilizing the thermally responsive aliphatic partition and magnetic bead combination, as described in the present disclosure. FIG. 3 demonstrates this sample-to-answer SARS-CoV-2 spike protein antibody detection in whole blood.

To perform this method, 1 μm streptavidin magnetic beads (from Pierce) were prepared by gathering 100 μL 10 mg/mL beads to the side of a tube using an external magnet, aspirating out their buffer, and rinsing the beads with 200 μL of wash buffer, comprising 25 mM Tris and 150 mM NaCl (both from Sigma-Aldrich). The beads were gathered to the side of the tube using an external magnet, and the wash buffer was removed. 50 μL 200 μg/mL SARS-CoV-2 biotinylated spike RBD protein (ProSci) was added to the 1 mg washed beads and left to incubate for 1 hour at room temperature at or between 20° C. and 25° C. Following this incubation, the beads were washed three times by magnetically gathering them to the side of the tube, aspirating out the supernatant, and washing them with 200 μL of wash buffer. On the final rinse, 25 μL of 0.1 M phosphate buffer was added to the mass of beads, resulting in 40 mg/mL magnetic beads coated in SARS-CoV-2 spike RBD protein.

To prepare the test assembly cartridge, cartridges with channels (3×3×47 mm³) were 3D printed with Prusament UV sensitive resin from Prusa Research. Once cured, a coverslip (Fisher Scientific) was glued to the open face of the cartridge, covering the channel, and left to dry overnight at room temperature. To ensure a hydrophobic surface, 423 μL of glass water repellent (Rain-X®) was incubated in the cartridge for 30 minutes at room temperature. Following incubation, excess glass water repellent was removed and the cartridges were washed three times with water.

To prepare the aliphatic partitions, eicosane (Tm=42° C.), a higher order alkane, was used to form the TRAPs to separate each region or sub-region. First, the cartridge channels were filled with 50 μL solution containing 5 μM Amplex Red (Biotium) and 1 mM hydrogen peroxide (Fisher Scientific). To prepare the eicosane (Alfa Aesar), it was first melted by placing it in a glass vial on a hot plate at 120° C. The 30 μL of melted eicosane quickly hardened as it was deposited atop the Amplex Red/hydrogen peroxide layer. Then 60 L 0.1 M phosphate buffer was added, followed by another 30 μL of melted eicosane, followed by 60 μL 0.1 M phosphate buffer, followed by 30 μL of melted eicosane, followed by 50 μL 100 ng/mL horseradish peroxidase (HRP)-conjugated anti-rabbit IgG antibodies (ThermoFisher), followed by 30 μL of melted eicosane. Finally, in the top zone, 2.5 μL 40 mg/mL magnetic beads coated in SARS-CoV-2 spike RBD protein was added.

To prepare the sample, whole blood was withdrawn from an exposed vessel at the elbow pocket of swine forelimbs within 15 minutes after the animal was euthanized. The blood was well mixed with an EDTA-coated collection tube and subsequently stored at 4° C. Immediately preceding the experiment, the blood was spiked with varying concentrations (0-1000 ng/mL) of SARS-CoV-2 spike RBD protein antibodies (ThermoFisher). 50 μL spiked blood samples were added to the top zone of the TRAP assay.

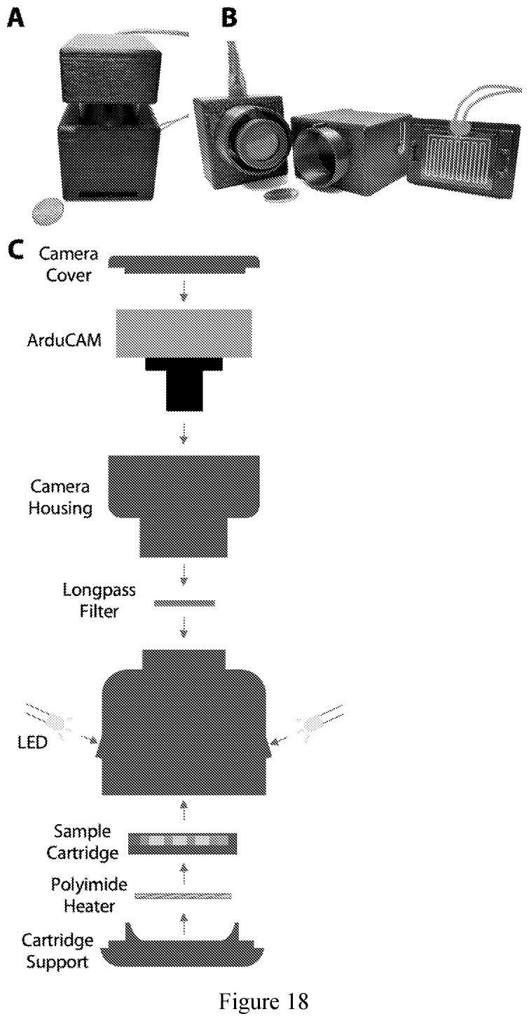

Fluorescence measurements were taken by placing a cartridge into a portable fluorescence reader as depicted in FIG. 18. The fluorescence reader is made up of 3D printed parts that secure in place an ArduCAM MT9M001 Camera with an ArduCAM USB2 Camera Shield (ArduCAM), a 570 nm longpass filter (Thorlabs) on the lens of the camera, two 525 nm LEDs to excite the sample, and a polyimide heater, shown assembled in FIG. 18A. As shown in FIG. 18B, the device opens to separate the heater from the ArduCAM to insert an assay cartridge. The ArduCAM is housed behind a longpass filter to detect fluorescence produced from a sample, which is excited by LEDs built into the device above the sample cartridge, as shown in FIG. 18C. The cartridge is placed on top of a polyimide heater to liquefy the TRAPs. The support in which the sample cartridge sits positions the sample in a precise location relative to the camera.

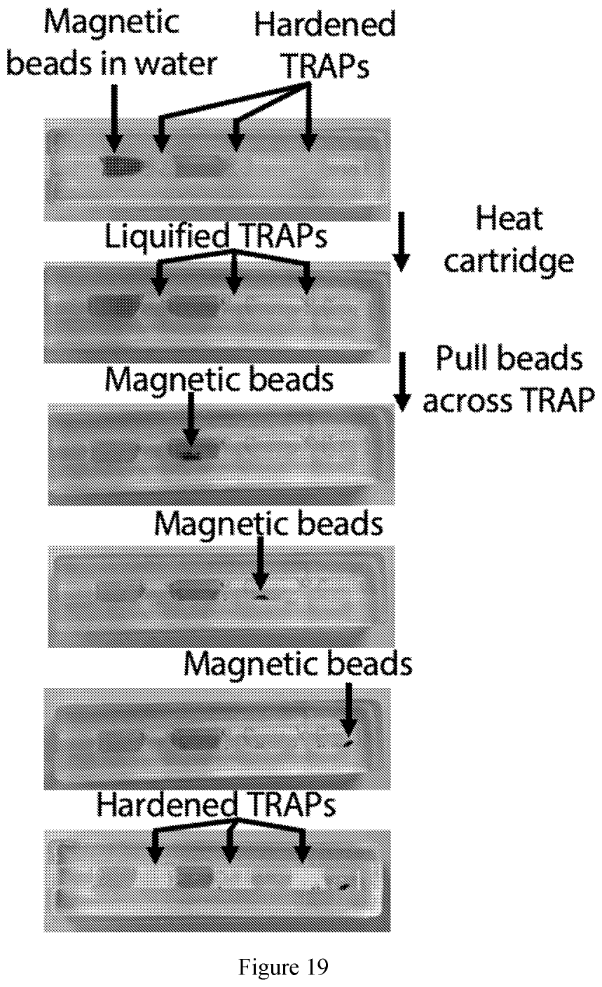

After the blood sample is added to the top zone of the assay, the cartridge was placed on the portable heater to melt the eicosane layers. The heat required to melt eicosane in each device was supplied via a polyimide heating pad that adhered to the 3D printed support that holds the sample cartridge in place (as seen in FIG. 18C). Upon placing a sample cartridge into its support, 6V of DC power was supplied to the polyimide heater. About two minutes after switching the power on, the temperature of the heater reaches eicosane's melting point. Once melted, an external magnet was used to apply a magnetic field the beads and move the beads across the first layer of eicosane, into the region containing HRP-conjugated antibodies. The external magnet was pulled at a rate of 0.2 mm/s to 10 mm/s. The heater was turned off and the beads were left to incubate for 30 minutes. After incubating, the heater was turned back on to melt the eicosane layers. Then, an external magnet was used to apply a magnetic field the beads and move the beads across the second layer of eicosane, into one of the regions containing phosphate buffer (i.e., a rinse sub-region). The beads were subsequently pulled across the third layer of eicosane, into another region containing phosphate buffer. Finally, the beads were pulled across the fourth layer of eicosane, into the detecting region containing Amplex Red and hydrogen peroxide. The heater was turned off and the fluorescence was measured by the fluorescence reader 10 minutes after the beads reach the detecting region. FIG. 19 shows photos of magnetic beads moved through TRAPs in the cartridge.

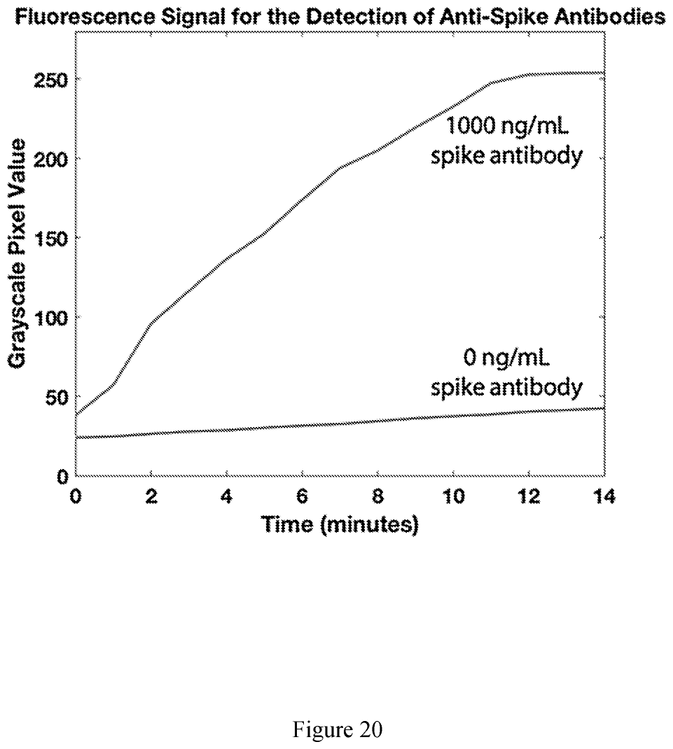

The results of the fluorescence quantification is shown in FIG. 20. FIG. 20 displays the fluorescence quantification of antibodies against SARS-CoV-2 spike proteins over a 14 minute period, where the heater was turned off at t=0 minutes.

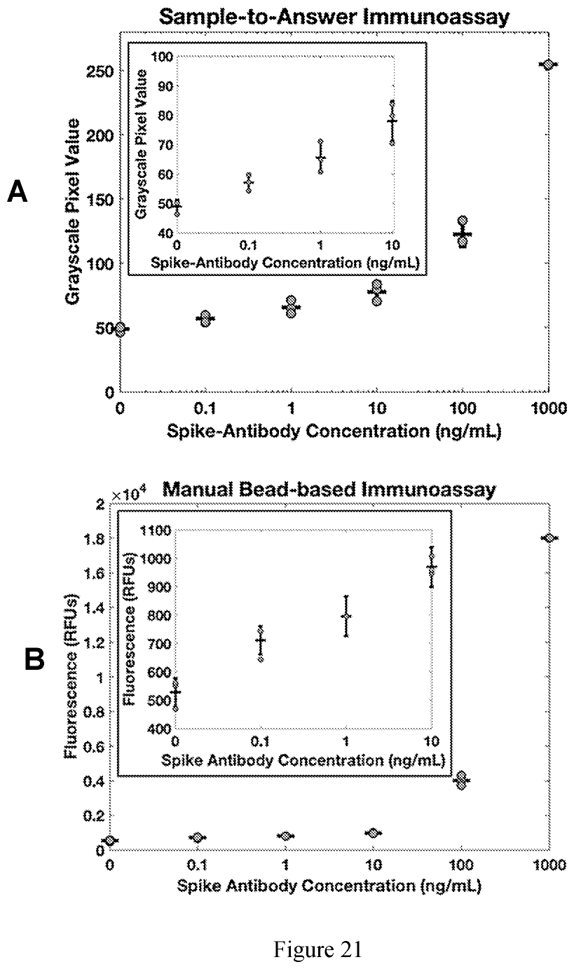

Using the International Union for Pure and Applied Chemistry (IUPAC) definition of the limit of detection (the concentration that generates a signal with a mean that is separated from the mean of the blank by three standard deviations of the blank), the limit of detection for this sample-to-answer assay was 84 μg/mL, as shown in FIG. 21A. The error bars in this figure are +1 standard deviation. Similarly, using the International Organization for Standardization (ISO) definition for the limit of detection (5% error on the blank and 5% error on a positive sample with the lowest concentration that is identifiable as positive), the limit of detection was 102 μg/mL. All values for detection limit were lower than the reported range of anti-spike antibodies in whole blood.

To compare data points, a manual bead-based ELISA assay with manual wash steps was performed by adding 2.5 μL 40 mg/mL magnetic beads coated in SARS-CoV-2 spike RBD protein to 50 μL whole blood samples spiked with SARS-CoV-2 spike RBD protein antibodies (0-1000 ng/mL). The beads and antibodies were left to incubate at room temperature for 30 minutes. The beads were gathered to the side of the tube using an external magnet, the supernatant was aspirated out, and they were rinsed with 200 μL of wash buffer three times. On the final rinse, the beads were re-suspended in 50 μL 100 ng/mL HRP-conjugated anti-rabbit IgG antibodies and left to incubate at room temperature for 30 minutes. The beads were gathered to the side of the tube using an external magnet, the supernatant was aspirated out, and they were rinsed with 200 μL of wash buffer three times. On the final rinse, the beads were re-suspended for 5 minutes in 5 μL elution buffer, which was comprised of 0.1 M glycine (Sigma-Aldrich) at pH 2. 5 μL of supernatant was collected and added to a 50 μL solution containing 5 μM Amplex Red and 1 mM hydrogen peroxide. Fluorescence resulting from manual bead washing was measured using a Synergy LX plate reader from BioTek (530 nm excitation, 590 nm emission). After the eluted sample was added to the Amplex Red/hydrogen peroxide solution, the liquid was moved into a 96-well plate. A fluorescence measurement was taken after 10 minutes.

Using the IUPAC definition, the limit of detection of this manual bead-based assay is 68 pg/mL, comparable to the example sample-to-answer assay (84 pg/mL). Likewise, using the ISO definition, the limit of detection of the manual bead-based assay is 80 pg/mL, similar to the performance of the example sample-to-answer assay (102 pg/mL). Both methods resulted in comparable limits of detection, suggesting the example sample-to-answer assay does not sacrifice sensitivity as it takes on key elements of point-of-care diagnostics.

FIG. 21B displays the fluorescence quantification of antibodies against SARS-CoV-2 spike proteins in whole blood using a bead-based immunoassay with a manual rinse steps and a benchtop plate reader (N=3). FIG. 16B displays the detection limit as 68 pg/mL using the IUPAC definition. The error bars are ±1 standard deviation.

To be truly point-of-care, assays need to be sample-to-answer, implying that whole blood samples must be collected and loaded into the cartridge without any precise manual sample transfers. Thus, point-of-care tests cannot rely on venous blood draws performed by phlebotomists and should instead enable the patient to draw their own sample or enable easy collection at a collection site via nurse, lab technician, or physician's assistant. The device used in this example was able to pull a precise volume of whole blood directly from finger prick into the cartridge via a capillary tube. The capillary tube was built into the cap (FIG. 5A), which when contacted with the blood sample (FIG. 5B), quickly wicked up blood (FIG. 5C) until a precise volume was reached (FIG. 5D). The channel was then tapped to empty the blood into the cartridge (FIG. 5E) and the capillary tube cap was exchanged with a sealed cap after the sample was loaded (FIG. 5F). This entire process was completed in 165 seconds. The integration of this sample collection step was crucial for sample-to-answer diagnostics as there are no manual sample preparation steps required of the user.

This example demonstrated a sample-to-answer assay for the detection of anti-spike antibodies in whole blood. Also described was a detection limit below the clinical threshold cutoff to be considered positive for antibodies against the SARS-CoV-2 spike protein. Fluorescence measurements were taken using a portable reader containing a built-in heater to integrate sample preparation steps into the overall system. Finally, this example has also integrated the blood collection step using a built-in capillary tube.

### Example 4

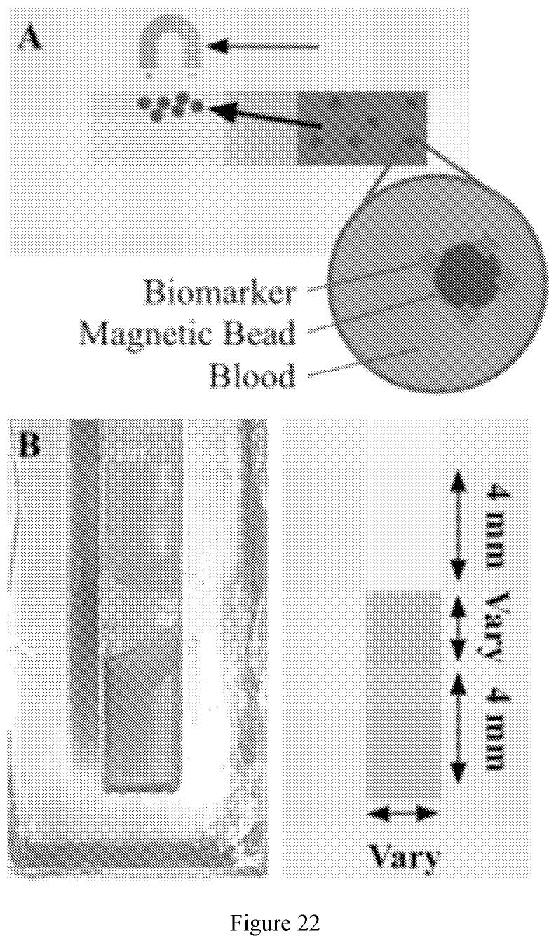

To determine the behavior of alkane in a small channel, experiments were conducted in 3D-printed channels. A range of channel sizes both hydrophobic (native resin) and hydrophilic (resin modified with fetal bovine serum) with square cross-sections were filled such that a layer of eicosane wax separated two 4 mm layers of dyed water as seen in FIG. 22. FIG. 22A illustrates a schematic application of the TRAP in which magnetic beads pull biomarkers out of a blood layer through melted alkane into a rinse zone. FIG. 22B shows a photo and schematic of a resin channel showing two dyed water layers separated by a TRAP. The channel was placed on a 60° C. hot plate either vertically or horizontally. A thinner layer of wax would break, which caused the two dyes to mix, while a thicker layer of wax kept the two dyes separated. The thickness of the wax layer (plug length) was varied to experimentally find the breakage threshold for several different sizes of channels. The geometric model equations are displayed in Example 1.

In the second experiment, magnetic beads were introduced to the system. In a horizontal 3×3 mm channel with a 2 mm thick wax layer, magnetic beads and FAM fluorophores were added to the layer on top of the wax. After melting the wax on a 60° C. hot plate, a magnet was used to move the beads across the TRAP. To verify that no leakage occurred during transfer, fluorescence measurements were taken before and after the transfer by a portable fluorescence reader.

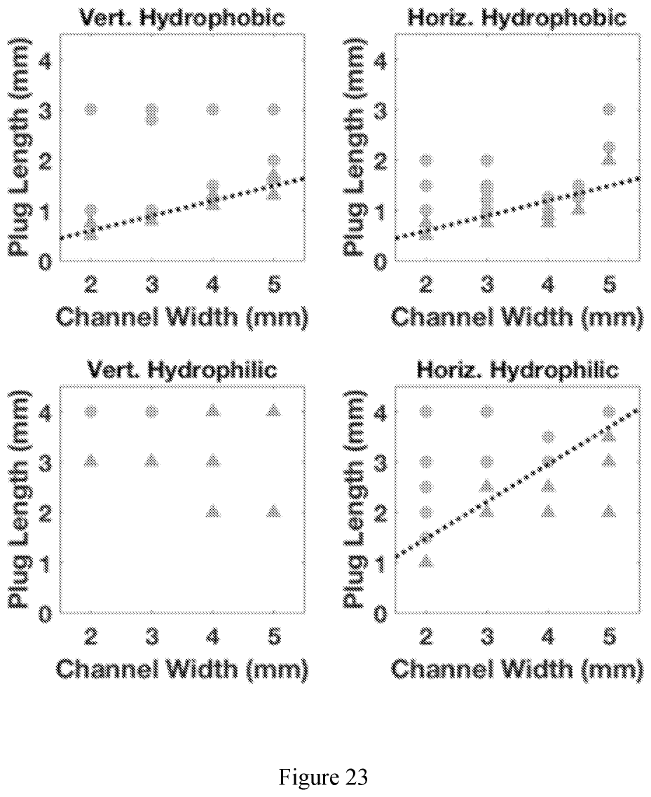

Because of the buoyancy of the alkane, TRAPs in vertical and horizontal configurations were investigated. FIG. 23 depicts the results of several combinations of plug length and channel width for vertical or horizontal configurations and hydrophobic or hydrophilic channels, where green circles indicate that the TRAP stayed, and red triangles indicate that the TRAP broke and dyes mixed. In the vertical case, it was determined that as the alkane liquefies, it is drawn to the walls of the hydrophobic resin channel, pinching in the center. Based on the plug length, channel width, and contact angle of the water-alkane-resin interface, a geometric model was produced (plotted as a dotted line in the top-left panel of FIG. 23) to predict if this pinching phenomenon results in a static or removable partition. Similar behavior is seen when the same channel is horizontal; however, this model broke down when the impact of gravity changed the behavior in channel widths around 5 mm (as seen in the top-right panel of FIG. 23).

To implement an assay in which mixing of reagents (i.e., TRAP breakage) is desired, a thin (1 mm) plug length could be used. However, in a hydrophilic channel, gravity dominates and a TRAP is more likely to break, allowing two solutions to mix. In applications where static partitions are desired, hydrophobic channels should be used while hydrophilic channels should be used when removable partitions are desired.

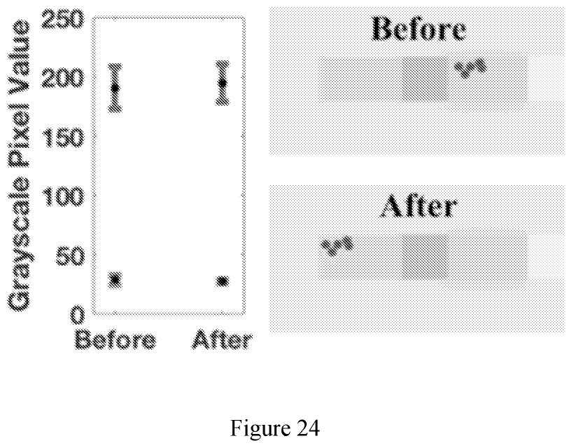

In various embodiments, functionalized magnetic beads are pulled through a static aliphatic partition that has been liquefied. To evaluate this diagnostic assay, the robustness against leakage after beads are moved across a TRAP was investigated. FIG. 24 shows fluorescence measurements of the water layer (red) and fluorescence layer (blue) along with their corresponding schematics of a channel before and after beads were pulled through a TRAP via a magnet. The result of this experiment showed that minimal leakage of the fluorophore through the partition occurred when transferring beads.

TRAPs can serve as low-cost automated valves that can be applied to systems where reagent manipulation is done without user interaction. The predictable behavior of leak-free partitions permeable to magnetic beads along with partitions that can be removed to allow solutions to mix at a specified time.

Although the present disclosure has been described with respect to one or more particular embodiments and/or examples, it will be understood that other embodiments and/or examples of the present disclosure may be made without departing from the scope of the present disclosure.

1. A method for detecting the presence of an analyte comprising:
i) contacting a sample suspected of containing the analyte together or separately with i) a plurality of magnetic beads having a plurality of first capture molecules attached thereto, wherein the first capture molecules have a specific affinity for the analyte, and ii) a plurality of second capture molecules having a detectable label attached thereto, wherein the second capture molecules have a specific affinity for the analyte, to form complexes, each complex comprising the analyte bound to the first capture molecule and the second capture molecule;
ii) heating one or more solid aliphatic partitions to a temperature of 40° C. to 65° C., such that the one or more solid aliphatic partitions liquefy;
iii) separating the complexes from unbound second capture molecules by selectively moving the complexes through the one or more liquefied aliphatic partitions via application of a magnetic field; and
iv) detecting and optionally quantifying the signal generated from the detectable labels of the second capture molecules of the separated complexes;
wherein the presence of a detectable signal is indicative of the presence of the analyte and the magnitude of the signal is indicative of the amount of the analyte in the sample.

2. The method of claim 1, wherein the sample is a biological sample.

3. The method of claim 2, wherein the biological sample is chosen from whole blood, blood fractions, plasma, serum, saliva, urine, stool, sweat, mucous, tears, breast milk, semen, tissue, placental tissue, conditioned medium, tissue culture medium, and bone marrow.

4. The method of claim 1, wherein the analyte is an antibody or an antigen.

5. The method of claim 4, wherein the antibody is directed to a pathogenic antigen.

6. The method of claim 5, wherein the pathogenic antigen is a microbial antigen, a bacterial antigen, or a viral antigen.

7. The method of claim 6, wherein the viral antigen is associated with SARS-CoV-2, Hepatitis C, Epstein-Barr, Zika, Ebola, Herpes simplex, Norovirus, Influenza, or Chikungunya.

8. The method of claim 1, wherein the plurality of second capture molecules have horseradish peroxidase attached thereto.

9. The method of claim 1, wherein the sample is sequentially contacted with the plurality of magnetic beads having the plurality of first capture molecules attached thereto and the plurality of second capture molecules having the detectable label attached thereto.

10. The method of claim 9, wherein the sample is rinsed after contacting the plurality of magnetic beads having the plurality of first capture molecules attached thereto and prior to contacting the plurality of second capture molecules having the detectable label attached thereto.

11. The method of claim 1, wherein the one or more aliphatic partitions comprise one or more alkanes.

12. The method of claim 11, wherein one or more alkanes are chosen from eicosane, docosane, hexacosane, heptacosane, nonococane, tetracosane, and octadecane.

13. The method of claim 1, wherein the magnetic field is generated by a magnet.

14. The method of claim 1, wherein the detectable signal is a fluorescence signal.

15. A testing system comprising:
a test assembly, the test assembly comprising an inlet configured to accept a sample comprising an analyte, a plurality of regions, and at least one solid aliphatic partition disposed therein, wherein adjacent regions of the plurality of regions are separated by one of the at least one solid aliphatic partition, and the plurality of regions includes a binding region and a detecting region, and the at least one solid aliphatic partition has a melting point of 40° C. to 65° C.;
wherein the binding region comprises a plurality of magnetic beads having a plurality of first capture molecules attached thereto, wherein the first capture molecules have a specific affinity for the analyte and a plurality of second capture molecules having a detectable label attached thereto, wherein the second capture molecules have a specific affinity for the analyte,
wherein the detecting region comprises a detection composition configured to enable spectrophotometric measurement of the composition; and
a magnet configured to apply a magnetic field along a sequential path, whereby the plurality of magnetic beads move sequentially along the sequential path upon application of a magnetic force from the magnet, wherein the sequential path includes the binding region and the detecting region.

16. The testing system of claim 15, wherein the analyte is an antibody or an antigen.

17. The testing system of claim 16, wherein the antibody or the antigen is associated with SARS-CoV-2, Hepatitis C, Epstein-Barr, Zika, Ebola, Herpes simplex, Norovirus, Influenza, or Chikungunya.

18. The testing system of claim 15, wherein the at least one aliphatic partition comprises one or more alkanes.

19. The testing system of claim 18, wherein one or more alkanes are chosen from eicosane, docosane, hexacosane, heptacosane, nonococane, tetracosane, and octadecane.

20. The testing system of claim 15, wherein the at least one aliphatic partition, the binding region, and the detecting region are arranged in a horizontal hydrophobic channel or a vertical hydrophobic channel.

21. The testing system of claim 15, wherein the at least one aliphatic partition, the binding region, and the detecting region are arranged in a horizontal hydrophilic channel or a vertical hydrophilic channel.

22. The testing system of claim 15, wherein the at least one aliphatic partition is configured to separate the binding region and the detecting region.

23. The testing system of claim 15, wherein the binding region comprises a plurality of sub-regions, each adjacent region separated by one of the at least one aliphatic partitions.

24. The testing system of claim 23, wherein a first binding sub-region of the binding region comprises the plurality of magnetic beads attached to a plurality of first capture molecules, wherein the first capture molecule has a specific affinity for the analyte, and a second binding sub-region of the binding region comprises the plurality of second capture molecules attached to a detectable label, wherein the second capture molecule has a specific affinity for the analyte.

25. The testing system of claim 24, further comprising a rinsing sub-region disposed between the aliphatic partition of the first binding sub-region and the aliphatic partition of the second binding sub-region.

26. The testing system of claim 15, wherein the magnet is external to the test assembly.

27. The testing system of claim 15, wherein the magnet is configured to move across the test assembly along the sequential path.

28. The testing system of claim 15, wherein the magnet is disposed on an end of the test assembly and the magnetic field extends across the binding region and the detecting region sequentially.
